(19) United States
(12) Patent Application Publication
(10) Pub. No.: US 2022/0288151 A1
(43) Pub. Date: Sep. 15, 2022

(54) COMPOSITIONS AND METHODS FOR TREATING NEUROLOGICAL DISEASES

(71) Applicant: CARMEL HAIFA UNIVERSITY ECONOMIC CORPORATION LTD., Haifa (IL)

(72) Inventors: Yaacov ROZENBLUM, Zikhron Yaakov (IL); Elham TAHA, Kabul (IL)

(21) Appl. No.: 17/633,210

(22) PCT Filed: Aug. 5, 2020
(86) PCT No.: PCT/IL2020/050859

§ 371 Date: Feb. 6, 2022

Related U.S. Application Data
(60) Provisional application No. 62/882,675, filed on Aug. 5, 2019.

(51) Int. Cl. A61K 38/00; G01N 33/50; A61P 25/28

(52) U.S. Cl. CPC A61K 38/005; G01N 33/5058; A61P 25/28; G01N 2800/2821; G01N 2800/2835; G01N 2800/2857; G01N 2800/304

(57) ABSTRACT

The present invention relates to a method for treating a neurological disease in a subject including administering to the subject a pharmaceutical composition of a eukaryotic elongation factor 2 kinase (eEF2K)-inhibiting compound.

### CROSS-REFERENCE TO RELATED APPLICATIONS

This application claims the benefit of priority of U.S. Provisional Patent Application No. 62/882,675, titled “COMPOSITIONS AND METHODS FOR TREATING NEUROLOGICAL DISEASES”, filed Aug. 5, 2019, the contents of which are incorporated herein by reference in their entirety.

### FIELD OF INVENTION

The present invention is in the field of neurological diseases therapy.

### BACKGROUND

Throughout life, new neurons are generated and later incorporated into discrete brain regions, including the sub-granular zone (SGZ) of the hippocampal dentate gyms (DG), a brain structure sub-serving high-level cognitive abilities in the mammalian brain. These adult-born neurons, which originate from neuronal progenitor cells (NPC), differentiate and integrate into existing circuits to become fully functional neurons.

Neurogenesis in the adult brain DG is inversely correlated with age and several neurological disorders, including Alzheimer's disease, depression, and epilepsy, but can also be enhanced in response to different forms of hippocampal-dependent learning, enriched environment, and/or physical activity.

### SUMMARY

According to one aspect, there is provided a method for treating a neurological disease in a subject in need thereof, the method comprising administering to the subject a pharmaceutical composition comprising therapeutically effective amount of a eukaryotic elongation factor 2 kinase (eEF2K)-inhibiting compound, thereby treating a neurological disease in the subject.

According to another aspect, there is provided a method for inducing neuron proliferation, the method comprising contacting the neuron with an effective amount of an eEF2K-inhibiting compound, thereby inducing neuron proliferation.

According to another aspect, there is provided a method of screening for a compound suitable for treating a neurological disease, the method comprising contacting a neuron with a compound, and determining activity of eEF2K in the presence of the compound, wherein reduction of eEF2K activity in the neuron in the presence of the compound compared to eEF2K activity in a neuron in the absence of the compound is indicative of the compound is suitable for treating a neurological disease.

In some embodiments, a pharmaceutical composition comprising an eEF2K-inhibiting compound, for use is the treatment of a neurological disease, is provided.

In some embodiments, the eEF2K-inhibiting compound inhibits eEF2K kinase activity.

In some embodiments, the eEF2K-inhibiting compound reduces the rate of eEF2 phosphorylation, the number of phosphorylated eEF2 molecules in a cell, or both.

In some embodiments, the eEF2K-inhibiting compound is selected from the group consisting of: nucleic acids, peptides, polypeptides, peptidomimetics, carbohydrates, lipids or other organic or inorganic molecules.

In some embodiments, the neuron is a neuron of the hippocampus.

In some embodiments, the neuron is a neuron of the dentate gyms (DG).

In some embodiments, the neuron is a mature excitatory neuron.

In some embodiments, the neuron is a neuron of a subject afflicted with a neurological disease.

In some embodiments, the neurological disease is selected from the group consisting of: Alzheimer's disease, Parkinson's disease, dementia, depression, epilepsy, memory loss, and cognitive impairment.

In some embodiments, the eEF2K-inhibiting compound is capable of inducing or promoting neuron proliferation.

In some embodiments, the eEF2K activity is ATP hydrolysis activity.

In some embodiments, the eEF2K activity is eEF2 phosphorylation.

In some embodiments, the phosphorylation is on Threonine 56 of the eEF2.

In some embodiments, the eEF2K activity is protein translation inhibition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1P are images and graphs showing that the eukaryotic elongation factor 2 kinase (eEF2K)/eEF2 pathway regulates neurogenesis and dentate gyms (DG)-dependent behavior in the mature brain. (1A) Non-limiting example of an outline of the experiment and steady state deep proteomics analysis. (1B) A volcano plot showing the differentially expressed proteins with Log 2 fold difference, ±15% cutoff, and p value between eEF2K-Knock out (KO) and Wild type (WT) mice. (1C) a hippocampal tissue was treated as in 1A and then subjected to enrichment analysis. Proteins that significantly changed (p<0.05, 354 proteins) were classified by using the online bioinformatics source portal gene ontology (geneontology.org) according to their involvement in biological process (see FIG. 6B), and 53 neurogenesis-related proteins were enriched in eEF2K-KO mice. (1D) Validation of dentate gyms (DG) punches was done by examining prox1 protein levels normalized to tubulin in the dentate gyms and cortex. Prox1 was enriched in the DG lysate compared to cortex lysate. Means ±SEM are shown (n=6-7, *p<0.0001). Full length immunoblots from DG and cortical tissue (and their quantifications) are shown in FIG. 6C. (1E) Decorin protein expression levels were significantly higher in the DG of eEF2K-KO mice compared to WT mice. Means ±SEM are shown (n=6, *p<0.05). Full length immunoblots from DG and cortical tissue (and their quantifications) are shown in FIG. 6G-6L. (1F) Vimentin protein expression levels were significantly higher in the DG of eEF2K-KO mice compared to WT mice. Means ±SEM are shown (n=10, *p<0.05). Full length immunoblots from DG and cortical tissue (and their quantifications) are shown in FIGS. 6G-6L. (1G) Upper panel: Representative immunoblots of Prox1 normalized to tubulin in the DG and cortex in WT mice. Lower panel: Representative immunoblots of decorin and vimentin normalized to tubulin in DG punches of eEF2K-KO and WT mice. (1H) Representative coronal hippocampal sections immunostained for BrdU from eEF2K-KO and WT littermate mice. Scale bar=20 μm, ×40. (1I) Quantification of BrdU positive cells (*p<0.0001; n=8). eEF2K-KO mice show higher levels of BrdU positive cells. (1J) Representative coronal hippocampal sections immunostained for DCX from WT and eEF2K-KO mice. Scale bar=20 μm, ×40. (1K) Quantification of DCX positive neurons (*p<0.0001; n=8). eEF2K-KO mice showed significantly higher levels of immature neurons in the dentate gyms. (1L) Non-limiting experimental design to test discrimination between two highly similar contexts A and B. Mice were conditioned in context A for three constitutive days. Discrimination for A, B, C, less similar, and D, different context, was examined on day 4. Mice were exposed to context A and 2 h later to context B on day 18 (test 2) and day 32 (test 3). (1M-1P) Analysis of freezing levels in context A for three days. eEF2K-KO mice and WT mice had similar levels of freezing in context A acquisition (n=11-13). On day 4, the mice were exposed to the four contexts. eEF2K-KO mice showed significantly better discrimination between context A and B (highly similar contexts) than WT mice. Both groups were able to recognize C and D. On day 18 and 32, the mice were tested for discrimination between context A and B. eEF2K-KO mice maintained their better discrimination between A and B compared to WT mice and their memory strength was better. Discrimination index per day is shown in FIGS. 6M-6O.

FIGS. 2A-2L are images and graphs showing that enriched environment, voluntary exercise, and ketamine, induce eEF2 dephosphorylation and hippocampal neurogenesis in the mature brain. (2A) A non-limiting experimental design. One group of mice was exposed to an enriched environment, which includes toys and a running wheel, and the second (control) group control had no such enrichment. Three times a week for four weeks mice received an i.p. injection of either temozolomide (TMZ) 20 mg/kg or vehicle. On day 29, the mice were allocated to immunohistochemistry (IHC) and western blot (using DG punches) groups. (2B) Representative coronal hippocampal sections immunostained for BrdU from WT mice, four groups: 1. Not Enriched/vehicle; 2. Not Enriched/TMZ; 3. Enriched/vehicle 4. Enriched/TMZ. Scale bar=20 μm, ×40. (2C) Quantification of BrdU positive cells (*p<0.0001; n=8) in the different groups. Enriched environment induced an increase in BrdU positive cells, which was blocked by TMZ. (2D) Representative coronal hippocampal sections immunostained for DCX from the four different groups. Scale bar=20 μm, ×40. (2E) Enriched environment induced an increase in DCX positive neurons, which was blocked by TMZ (p<0.0001; n=8). (2F) Representative immunoblots of pThr56eEF2 (peEF2) normalized to total eEF2 and tubulin in the DG lysates of enriched environment and control mice. (2G) Enriched environment induces eEF2 dephosphorylation in dentate gyms lysates compared to control. Means ±SEM are shown (n=8-9, *p<0.05). Full-length immunoblots from DG and cortical tissue (and their quantifications) are shown in FIGS. 7A-7E. (2H) Representative immunoblots of pThr56eEF2 normalized to total eEF2 and tubulin in the DG lysates of 5 mg/kg ketamine and saline-injected mice. (2I) Ketamine administration (5 mg/kg) induces eEF2 dephosphorylation in the dentate gyms. Means ±SEM are shown (n=9, *p<0.05). Full length immunoblots from DG and cortical tissue (and their quantifications) are shown in FIGS. 7I-7L. (2J) A micrograph of immunoblots for pAMPK, AMPK, and tubulin in DG lysates of mice subjected to an enriched environment and of control mice. (2K) A graph showing that the enriched environment induces dephosphorylation of T172 in AMPK in mouse DG lysates compared with that in control. Mean ±SEM is shown (t test; n=8-9; p<0.05). (2L) A schematic representation of enriched environment-responsive signaling pathway assessed in this study. There was no change in mTOR pathway via the S6K1 or ERK pathway, signaling pathways that are upstream of eEF2K. the main input regulating eEF2K in an enriched environment is via AMPK, an energy sensor in the brain. Likewise, ketamine induces dephosphorylation of eEF2K and to the dephosphorylation of eEF2K on Thr-56 and upregulation of neurogenesis and neurogenesis-related proteins. Dashed lines indicate no effect on eEF2K.

FIGS. 3A-3V are images and graphs showing that reduced expression levels of eEF2K in DG excitatory neurons enhance neurogenesis and hippocampal-dependent behavior in adult mice. (3A) A non-limiting experimental design. Two months old mice were bilaterally CaMKII GFP/CRE viruses injected in the DG of the hippocampus. After 6-7 weeks, the mice underwent behavioral analysis. The mice were sacrificed for IHC-neurogenesis analysis and biochemical analysis (using DG punches). (3B) Stereotaxic injection of AAV CaMKII GFP/Cre vectors targeting adult excitatory neurons in the DG. The coordinates are: AP −2.00 mm, ML±1.3 mm, DV −1.9 mm. Scale bar: 50 μm, magnification ×20. (3C) Representative immunoblots of pThr56eEF2 normalized to total eEF2 and tubulin in DG punches of CaMKII-Cre-injected compared to CaMKII-GFP-injected mice. (3D) Reduced pThr56eEF2 protein levels (when normalized to total eEF2) in the DG of CaMKII-Cre-injected mice compared to CaMKII-GFP-injected mice (*p<0.05, n=11-12). Full length immunoblots of pThr56eEF2, eEF2, vimentin, decorin, tubulin, and their quantification are shown in FIGS. 8A-8E. (3E) Representative coronal hippocampal sections immunostained for BrdU from CaMKII-GFP- and CaMKII-Cre-injected mice (n=9). Scale bar=20 μm, ×40. (3F) Quantification of BrdU positive cells (*p<0.01; n=9) in the different groups. Reduced expression of eEF2K in excitatory DG neurons in eEF2K foxed mice increased BrdU positive cells, which was blocked by TMZ. (3G) Representative coronal hippocampal sections immunostained for DCX from CaMKII-GFP-injected and CaMKII-Cre-injected mice. Scale bar=20 μm, ×40. (3H) Quantification of DCX positive neuron population. CaMKII-Cre-injected mice showed higher DCX levels compared to CaMKII-GFP injected controls (n=9, *p<0.0001). (3I) Analysis of freezing levels in context A for three days. CaMKII-Cre-injected and CaMKII-GFP-injected mice had similar levels of freezing in context A acquisition (p>0.05, n=12). (3J) Discrimination tests. On day 4, the mice were exposed to the four contexts. CaMKII-Cre-injected mice show significantly better discrimination between context A and B (highly similar contexts) than CaMKII-GFP-injected mice (*p<0.001, n=12). Both groups were able to recognize C and D. Discrimination index analysis is shown in FIG. 8G. (3K) Analysis of freezing levels in context A and B on day 18. CaMKII-Cre-injected mice showed significantly better discrimination between context A and B compared to CaMKII-GFP-injected mice (n=12, *p<0.001). Discrimination index analysis is shown in FIG. 8H. (3L) Analysis of freezing levels in context A and B on day 32. CaMKII-Cre-injected mice showed significantly better discrimination between context A and B compared to CaMKII-GFP-injected mice and their memory strength is better (n=12, *p<0.001). Discrimination index analysis is shown in FIG. 8I. (3M) CaMKII-Cre-injected mice exhibited better latencies to locating the hidden platform across training days in a weak protocol of MWM (*p<0.0001, n=6 mice). (3N) CaMKII-Cre-injected mice showed a modest preference for the target quadrant during the probe trial on day 4 (PT1) compared to CaMKII-GFP-injected mice (p=0.070, n=6). Time spent in each quadrant on PT2 is shown in FIG. 8J. (3O) One cue test of pattern completion was performed two weeks following the last probe test (PT2). CaMKII-Cre-injected mice found the hidden platform in one cue test faster than CaMKII-GFP-injected mice (*p<0.05, n=6). Full cue test analysis after one cue test is shown in FIGS. 8K-8L. (3P) Antidepressant-like behavior was better in CaMKII-Cre-injected mice compared to CaMKII-GFP-injected mice (*p<0.01, n=6-7). Antidepressant behavior was examined using the forced swim test. WT mice were injected 5 mg/kg ketamine as positive control. Immobility time was manually scored for 4 min in a glass beaker filled with warm water. (3Q) A vertical bar graph showing the percentage overlap of GFP and DCX. 96.16% and 89.3% of DCX-positive neurons in CaMKII-Cre and CaMKII-GFP-injected mice, respectively, observed, are not GFP-positive cells in DG (Mann-Whitney U test; n=4; p<0.05). (3R-3S) Graphs summarizing an open field analysis. CaMKII-Cre-injected mice traveled significantly more in the open arena and made more crossings in the center zone. Mean ±SEMS is shown (3R, t test, n=6, p<0.01; 3S, Mann-Whitney U test, n=6, p<0.05). (3T) novel object exploration analysis. CaMKII-Cre-injected mice explored significantly more novel object than did CaMKII-GFP-injected mice. Mean ±SEM is shown (t test; n=6; p<0.05). (3U) Special interaction test analysis. CaMKII-Cre-injected mice show significantly higher sociability index than CaMKII-GFP-injected mice. Mean ±SEM sis shown (t test; n=5-6; p<0.05). Sociability index was calculated as stranger1 exploration/total exploration (stranger1+empty wire cup). (3V) CaMKII-Cre-injected mice and CaMKII-GFP-injected mice show similar social novelty interaction index. Mean ±SEM is shown (t test; n=5-6; p>0.05). Novelty social interaction index was calculated as novel stranger exploration/total exploration (stranger1+novel stranger).

FIGS. 4A-40 are image and graphs showing that downregulation of eEF2K in the DG of old mice enhances neurogenesis and DG-dependent behavior. (4A) Analysis of freezing levels in context A for three days. eEF2K-KO and WT littermate old mice have similar levels of freezing in context A acquisition (*p>0.05, n=9). (4B) Discrimination tests. On day 4 the mice were exposed to the four contexts. eEF2K-KO old mice showed better discrimination between context A and B (highly similar contexts) than WT old mice (*p<0.01, n=9). Both groups were able to recognize C and D. Discrimination index analysis is shown in FIG. 9A. (4C) Analysis of freezing levels in context A and B on day 18. eEF2K-KO old mice showed significantly better discrimination between context A and B compared to WT old mice (*p=0.15, n=9). Discrimination index analysis is shown in FIG. 9B. (4D) Analysis of freezing levels in context A and B on day 32 eEF2K-KO old mice showed significantly better discrimination between context A and B compared to WT old mice (*p<0.05, n=9). Discrimination index analysis is shown in FIG. 9C. (4E) A non-limiting experimental design. Aged mice (14 months old) were bilaterally injected with CaMKII GFP/Cre viruses into the DG of the hippocampus. After 6-7 weeks, the mice underwent context discrimination. The mice were sacrificed for IHC neurogenesis analysis. (4F) Representative coronal hippocampal sections immunostained for BrdU from aged CaMKII GFP/Cre-injected mice are shown. Scale bar=20 μm, ×40. (4G) Quantification of BrdU positive cells (*p<0.05; n=9) in the different groups. Reduced expression of eEF2K in excitatory neurons in DG of old eEF2K foxed mice increased the number of BrdU positive cells (n=9, *p=0.001). (4H) Representative coronal hippocampal sections immunostained for DCX from CaMKII-GFP-injected and CaMKII-Cre-injected old mice. Scale bar=20 μm, ×40. (4I) Quantification of DCX positive neuronal population. CaMKII-Cre-injected mice showed higher levels of DCX positive neurons compared to CaMKII-GFP-injected old mice (n=9, p<0.0001). (4J) Old CaMKII-Cre injected mice and CaMKII-GFP-injected mice travelled a similar distance in open field arena (p>0.05, n=6). (4K) Old CaMKII-Cre-injected mice spent significantly more time in the center zone of the open field (*p<0.05, n=6). (4L) Analysis of freezing levels in context A for three days. CaMKII-Cre-injected and CaMKII-GFP-injected old mice had comparable levels of freezing in context A acquisition (p>0.05, n=12). (4M-4O) Discrimination tests. On day 4, the mice were exposed to the four contexts. Old CaMKII-Cre-injected mice showed significantly better discrimination between context A and B (highly similar contexts) than CaMKII-GFP injected old mice. Both groups were able to recognize C and D. On day 18 and 32, the mice were tested for context A and B. CaMKII-Cre-injected mice maintained their discrimination between A and B compared to CaMKII-GFP-injected old mice and their memory strength was better (*p=0.017, n=12). Discrimination index analysis for three tests is shown in FIGS. 9D-10F.

FIG. 5 is a non-limiting scheme showing that the eEF2K/eEF2 pathway is the key molecular switch regulating hippocampal neurogenesis in the mature brain. A proposed molecular model for hippocampal neurogenesis induction via the eEF2K/eEF2 pathway is presented. Physical exercise, antidepressant drugs, and enriched environment stimulate neurogenesis in the dentate gyms of the hippocampus. Following the exposure to external neurogenic factors or gene manipulation targeting eEF2K, there is dephosphorylation of the eEF2 in the excitatory neurons of the dentate gyms, which leads to proteostasis changes in the hippocampus. This proteomic change in expression levels of neurogenesis-related proteins mediates the increase in neurogenesis that affects the following: cognitive functions, as tested in the context discrimination paradigm, spatial pattern completion, spatial memory tested using MWM, object recognition, and social behavior. In addition, anti-depressive behavior is better following neurogenesis up-regulation. Moreover, in vivo recording in the dentate gyms shows increased neuronal excitability, which leads to more inhibition at the population level in the dentate gyms. Targeting eEF2K specifically in dentate gyms excitatory neurons of old mice leads to increased neurogenesis and better memory. Overall, the emerging picture suggests that selectively targeting the eEF2K/eEF2 pathway in the excitatory neurons of the dentate gyms offers a novel strategy to enhance neurogenesis in the hippocampus.

FIGS. 6A-6O are images and graphs. (6A) A volcano plot with a 50% cutoff showing the differentially expressed proteins between eEF2K-KO and WT mice. There were more up-regulated proteins in eEF2K-KO mice compared to WT mice. (6B) Heat-map showing the normalized intensity of 54 proteins identified as neurogenesis-related proteins in eEF2K-KO compared to WT mice. (6C) Full length original immunoblots of proxy1 and tubulin are shown from dentate gyms (DG) and cortex lysates of WT mice. (6D) Full length original immunoblots of pThr56-eEF2, eEF2, and tubulin are shown in the DG of eEF2K-KO and WT mice. (6E) pThr56-eEF2 protein expression normalized to eEF2 was significantly reduced in the DG of eEF2K-KO (*p<0.0001, n=6). (6F) eEF2 protein level normalized to tubulin was not altered in DG lysate of eEF2K-KO compared to WT mice (p>0.05, n=6). (6G) Full length original immunoblots of decorin, vimentin, and tubulin in DG lysates of eEF2K-KO and WT mice. (6H) Full length original immunoblots of pThr56-eEF2, eEF2, and tubulin are shown in the cortex of eEF2K-KO and WT mice. (6I) pThr56-eEF2 protein expression normalized to eEF2 was significantly reduced in the cortex of eEF2K-KO (*p<0.0001, n=6). (6J) There was no change in eEF2 protein levels normalized to tubulin in cortex lysate of eEF2K-KO compared to WT mice (p>0.05, n=6). (6K) There was no significant change in decorin protein expression normalized to tubulin in cortex of eEF2K-KO compared to WT mice (p>0.05, n=6). (6L) Vimentin protein level was significantly reduced in the cortex of eEF2K-KO compared to WT mice (*p<0.05, n=6). (6M) Discrimination index analysis in eEF2K-KO and WT mice. Discrimination index was calculated as: (% freezing in context A-% freezing in context B)/(Total % of freezing in contexts A and B). eEF2K-KO mice had significantly better discrimination index between context A and B on day 4 (*p<0.01, n=12-13). (6N) eEF2K-KO mice and WT mice had similar discrimination index between context A and B on day 18 (p=0.052, n=12-13). (6O) eEF2K-KO mice had significantly better discrimination index between context A and B on day 32 (*p<0.05, n=12-13).

FIGS. 7A-7L are images and graphs. (7A) Uncropped original immunoblots of pThr56-eEF2, eEF2, and tubulin are shown from DG samples of enriched and control mice. (7B) There was no change in eEF2 protein level normalized to tubulin in enriched mice compared to control mice (p>0.05 n=8-9). (7C) Uncropped original immunoblots of pThr56-eEF2, eEF2, and tubulin are shown from cortex samples of enriched and control mice. (7D) There was no significant change in pThr56eEF2 protein levels normalized to eEF2 in enriched mice compared to control mice in cortex tissue (p>0.05 n=8-9). (7E) There was no change in eEF2 protein level normalized to tubulin in enriched mice compared to control mice in cortex tissue (p>0.05 n=8-9). (7F) Uncropped original full length immunoblots of pThr172AMPK, AMPK, and tubulin from DG samples of enriched and control mice. (7G) pThr172AMPK normalized to AMPK protein in DG samples. Enriched environment induced lower levels of pThr172AMPK normalized to AMPK in enriched mice compared to control mice (*p=0.05 n=8-9). (711) AMPK normalized to tubulin was unchanged in enriched mice compared to control mice (p>0.05 n=8-9). (7I) Full length original immunoblots of pThr56-eEF2, eEF2, and tubulin from DG samples collected 30 min after saline/ketamine (5 mg/kg) i.p. injection. (7J) Full length original immunoblots of pThr56-eEF2, eEF2, and tubulin from cortical samples collected 30 min after saline/ketamine (5 mg/kg) i.p. injection. (7K) There was no change in eEF2 protein levels normalized to tubulin in ketamine-injected mice compared to saline-injected mice (p>0.05 n=9). (7L) (Left) pT56eEF2 normalized to eEF2 protein in cortex tissue following 30 min of ketamine injection. Ketamine, 5 mg/kg, reduces pT56eEF2 levels in ketamine-injected mice compared to control (p=0.009 n=9). (Right) there was no change in eEF2 protein level normalized to tubulin in ketamine-injected mice compared to control mice in cortex tissue (p>0.05 n=9).

FIGS. 8A-8R are images and graphs. (8A) Full length original immunoblots of pThr56eEF2, eEF2, vimentin, decorin, mature-BDNF, and tubulin from DG samples of CaMKII-Cre-injected mice and-GFP-injected mice are shown. (8B) eEF2 protein level normalized to tubulin was similar in CaMKII-Cre-injected mice and CaMKII-GFP-injected mice (p>0.05, n=11-12). (8C) Vimentin normalized to tubulin was significantly up-regulated in DG in CaMKII-Cre-injected mice (p=0.002, n=11-12). (8D) Decorin normalized to tubulin was significantly up-regulated in DG in CaMKII-Cre-injected mice (p<0.05, n=6). (8E) Mature-BDNF normalized to tubulin was increased in the DG of CaMKII-Cre-injected mice (p<0.05, n=6). (8F) (Left) Fear conditioning acquisition analysis of naive eEF2K floxed/floxed mice (not injected) in context A. There was no difference between naive eEF2K floxed/floxed mice and CaMKII-GFP-injected mice in context A acquisition for three days. (Right) Context discrimination on day 4 between context A and B in Naive eEF2K floxed/floxed mice. There was no difference in context discrimination of A and B between naive eEF2K floxed/floxed mice and CaMKII-GFP-injected mice (n=7). (8G-8I) Discrimination index analysis on day 4, day 18, and 32 in CaMKII-Cre-injected mice and CaMKII-GFP-injected mice. CaMKII-Cre-injected mice showed significantly higher discrimination index for one month (p<0.0001, n=12). (8J) Probe test (PT2) analysis on day 6 of the Morris water maze. CaMKII-Cre-injected mice and CaMKII-GFP-injected mice spent significantly more time in the target quadrant compared to other quadrants (p<0.05, n=6). (8K) Probe test analysis of the Morris water maze with full cues after one cue test. This test was done 14 days following PT2. CaMKII-Cre-injected mice and CaMKII-GFP-injected mice spent significantly more time in the target quadrant compared to other quadrants (p<0.05, n=6). (8L) Latency to platform location/zone in full cue conditions after 14 days of PT2. CaMKII-Cre-injected mice and CaMKII-GFP-injected mice exhibited similar latencies to platform zone after 14 days (p>0.05, n=6). (8M) Reversal Morris water maze analysis. CaMKII-Cre-injected mice and CaMKII-GFP-injected mice show similar learning of the new platform location, suggesting normal cognitive flexibility (p>0.05, n=6). (8N-8O) Open field analysis. CaMKII-Cre-injected mice travelled significantly more in the open arena and made more crossings in the center zone (p<0.05, n=6 mice). (8P) Novel object exploration analysis in the open field paradigm. CaMKII-Cre-injected mice explored significantly more novel objects in the open field compared to CaMKII-GFP-injected mice (p<0.05, n=6). (8Q) Social interaction test analysis. CaMKII-Cre-injected mice showed significantly higher sociability index compared to CaMKII-GFP-injected mice (p<0.05, n=5-6). Sociability index was calculated as: stranger1 exploration/total exploration (stranger 1+empty wire cup). (8R) CaMKII-Cre-injected mice and CaMKII-GFP-injected mice showed similar social novelty interaction index (p>0.05, n=5-6). Novelty social interaction index was calculated as: novel stranger exploration/total exploration (stranger1+novel stranger).

FIGS. 9A-9F are graphs. (9A) Discrimination index analysis. There was no difference in discrimination index on day 4 between eEF2K-KO and WT old mice (p>0.05, n=9). (9B) eEF2K-KO old mice showed significantly higher discrimination index on day 18 (p<0.05, n=9). (C) There was no difference in discrimination index on day 32 between eEF2K-KO and WT old mice (p>0.05, n=9). (9D) There was no difference in discrimination index on day 4between CaMKII-Cre-injected mice and CaMKII-GFP-injected old mice (p>0.05, n=12). (9E) CaMKII-Cre-injected old mice show non-significant increase in discrimination index on day 18 compared to CaMKII-GFP-injected mice (p=0.065, n=12). (9F) There is no difference in discrimination index on day 32 between CaMKII-Cre-injected mice and CaMKII-GFP-injected old mice (p>0.05, n=12).

FIGS. 10A-10J include graphs showing strong stimuli reverse early LTP deficits in eEF2K KO mice. (10A) Basal synaptic transmission measured is normal in CaMKII-Cre-injected mice. Input-output relationship between CaMKII-Cre-injected mice and CaMKII-GFP-injected mice shows no significant difference with increasing stimulation intensities. Mean ±SEM is shown (two-way RM ANOVA; n=5; p>0.05). The basal circuitry properties of DG and EPSP slopes change of baseline were plotted against stimulus intensities of 60-300 μA. (10B) The population spike amplitude was not significantly different between genotypes across a range of stimulation intensities. Mean ±SEM is shown (two-way RM ANOVA; n=5; p>0.05). (10C) The EPSP-spike curve: compared with CaMKII-GFP-injected mice, CaMKII-Cre-injected mice show a slight shift of the E-S coupling curves, indicating that a larger PS was produced by a given fEPSP slope. EPSP-spike comparison shows no significant difference. Mean ±SEM is shown (three-way ANOVA; n=5; p>0.05). (10D) Representative sample field potential traces (mean of five consecutive responses) collected at baseline (black) and 1 h after HFS (gray). Scale bars, 2 mV and 5 mS. (10E) Times course plots of medical performant path-dentate gyms (DG)-evoked fEPSPs recorded before and after high-frequency stimulation (HFS) (indicated by arrows). Values are mean ±SEM of the maximum fEPSP slope expression as percentage of baseline. The magnitude of single 200-Hz-evoked E-LTP was comparable in both CaMKII-Cre-injected mice and CaMKII-GFP-injected mice. (10F-10G) include bar graphs representing the mean changes ±SEM in fEPSEP slope comparison at 40 min (t test; n=6; p>0.05; 10F and 10 min (t test; n=6; p>0.05; 10G) after HFS. (10H) Similarly, the magnitude of four 200-Hz-trains-evoked L-LTP in CaMKII-Cre-injected mice that decayed to baseline after 50 min and show phenotypic recovery at 180 min after HFS compared to CaMKII-GFP-injected mice. (10I-10J) include bar graphs representing the mean changes ±SEM in fEPSP slope comparison at 40 mine (t test; n=8; p<0.001; 10I) and 180 min (t test; n=8; p>0.05; 10J) after HFS.

FIGS. 11A-11E include a scheme, micrographs, and graphs. (11A) An experimental design. 2-3-month-old mice were scarified, and the hippocampi were extracted and used for subcellular tissue fractionation. Seven subcellular fractions were generated (see Methods) and the protein expression levels of KIF5A and NSF were analyzed using Western blot. (11B) Full length uncropped original immunoblots of KIF5A and tubulin are shown from the hippocampus of eEF2K-KO and WT mice. (11C) Full length uncropped original immunoblots of NSF and tubulin are shown from the hippocampus of eEF2K-KO and WT mice. (11D) KIF5A protein levels are significantly higher in the cytosolic (S3) fraction of the hippocampus of eEF2K-KO compared to WT mice (p=0.05, n=3). (11E) NSF protein levels are significantly higher in the synaptosomal (P2) fraction of the hippocampus of eEF2K-KO compared to WT mice (p=0.05, n=3).

FIGS. 12A-12E include vertical bar graphs. (12A-12B) graphs showing there was no change in BrdU (12A) and DCX (12B) levels in eIF2α-KI compared to WT mice (p>0.05; n=3-4). (12C) A discrimination index analysis in eEF2K-KO and WT mice. Discrimination index was calculated as: (% freezing in context A-% freezing in context B)/(Total % of freezing in contexts A and B). eEF2K-KO mice have significantly better discrimination index between context A and B on day 4 (p<0.01, n=12-13). (12D) eEF2K-KO mice and WT mice have a similar discrimination index between context A and B on day 18 (p=0.052, n=12-13). (12E) eEF2K-KO mice have a significantly better discrimination index between context A and B on day 32 (p<0.05, n=12-13).

FIGS. 13A-13F include micrographs and vertical bar graphs. (13A) Uncropped original full length immunoblots of pS6K1, S6K1, and tubulin from DG samples of enriched and control mice. (13B) pS6K1 normalized to S6K1 is unchanged in enriched mice compared to control mice (p>0.05, n=8-9). (13C) S6K1 normalized to tubulin is unchanged in enriched mice compared to control mice (p>0.05, n=8-9). (13D) Uncropped original full length immunoblots of pERK2, ERK2, and tubulin from DG samples of enriched and control mice. (13E) pERK2 normalized to ERK2 is unchanged in enriched mice compared to control mice (p>0.05, n=8-9). (13F) ERK2 normalized to tubulin is unchanged in enriched mice compared to control mice (p>0.05, n=8-9).

FIGS. 14A-14B include fluorescent micrographs and a vertical bar graph. (14A) Representative coronal hippocampal sections immunostained for BrdU from CaMKII-GFP- and CaMKII-Cre-injected mice (n=9). Scale bar, 50 μm, 20×. (14B) Quantification of BrdU positive cells (p<0.05; n=9). Reduced expression of eEF2K in excitatory DG neurons in eEF2K foxed mice increases the number of BrdU positive cells.

FIGS. 15A-15D include fluorescent micrographs and vertical bar graphs. (15A) Representative coronal hippocampal sections immunostained for BrdU from CaMKII-GFP- and CaMKII-Cre-injected mice treated with vehicle or TMZ for 6 weeks (See Methods), (n=3). Scale bar, 50 μm, 20×. (15B) Quantification of BrdU positive cells (p<0.05; n=3). Reduced expression of eEF2K in excitatory DG neurons in eEF2K floxed mice increases BrdU positive cells which was occluded using TMZ. (15C) Representative coronal hippocampal sections immunostained for DCX from CaMKII-GFP- and CaMKII-Cre-injected mice treated with Vehicle and TMZ for 6 weeks (See Methods), (n=4). Scale bar, 50 μm, 20×. (15D) Quantification of DCX positive cells (p<0.05; n=4). Reduced expression of eEF2K in excitatory DG neurons in eEF2K floxed mice increases DCX positive cells which was occluded by TMZ. CaMKII-GFP treated with TMZ showed non-significant reduced levels of DCX positive cells compared to CaMKII-GFP treated with vehicle.

FIGS. 16A-16B include fluorescent micrographs and a vertical bar graph. (16A) Representative coronal hippocampal sections immunostained for BrdU from Synapsin-GFP- and Synapsin-Cre-injected mice (n=5-6). Scale bar, 50 μm, 20×. (16B) Quantification of BrdU positive cells (p<0.01; n=5-6). Reduced expression of eEF2K in DG neurons in eEF2K floxed mice increases BrdU positive cells.

### DETAILED DESCRIPTION

Method

In some embodiments, a method for treating or preventing a neurological disease in a subject in need thereof, the method comprising administering to the subject a pharmaceutical composition comprising therapeutically effective amount of an eukaryotic elongation factor 2 kinase (eEF2K)-inhibiting compound, thereby treating a neurological disease in the subject, is provided.

In some embodiments, a method for inducing neuron proliferation, the method comprising contacting the neuron with an effective amount of an eEF2K-inhibiting compound, thereby inducing neuron proliferation, is provided.

In some embodiments, “induce” or “inducing” comprise promoting, propagating, enhancing, taking part in, being involved in, or any combination thereof.

In one embodiment, eEF2K inhibition reduces phosphorylation of the eEF2 protein. In one embodiment, eEF2K-inhibition reduces the rate of eEF2 phosphorylation, the number of phosphorylated eEF2 molecules, or both. In one embodiment, eEF2K inhibition increases concentration of an un-phosphorylated eEF2 protein. In another embodiment, eEF2K inhibition increases initiation of protein synthesis, elongation of protein synthesis, protein production rates, amount of protein per cell, cell proliferation, cell differentiation, cell survival, or combination thereof.

In some embodiments, eEF2K inhibition increases the number of proliferating cells, reduces the duration of cell proliferation (e.g., less time to complete the cell cycle and or/division), or a combination thereof.

eFF2K-Inhibiting Compound

In some embodiments, the eEF2K-inhibiting compound inhibits eEF2K kinase activity. In some embodiments, eEF2K activity is phosphorylation of eEF2K. In some embodiments, eEF2K activity is phosphorylation of Threonine 56 (Thr56e) of the eEF2.

As used herein, the term “Threonine 56” refers to the amino acid Threonine located in position 56 from the N′-terminal end of the eEF2 protein.

In some embodiments, eEF2K is a human eEF2K protein or peptide. In some embodiments, eEF2K comprises an amino acid sequence according to Accession number AAH32665.1.

In some embodiments, eEF2 is a human eEF2 protein or peptide. In some embodiments, eEF2 comprises an amino acid sequence according to Accession number AAH06547.1.

Methods for determining phosphorylation of a peptide, e.g., eEF2, are common and would be apparent to one of ordinary skill in the art. Non-limiting examples of methods for determining phosphorylation, include, but are not limited to, immunoassay using antibodies capable of binding either a phosphorylated form of a target protein or an unphosphorylated form of the target protein, expression of a target protein for expression in the presence of radioactive phosphate and determination of the rate/amount of phosphorylated proteins (e.g., pulse-chase), or any method known in the art.

As used herein, the term “kinase activity” encompasses catalysis of a reaction wherein a phosphate group is transferred from a high-energy donor (e.g., a phosphate-donating molecule) to a substrate molecule.

In some embodiments, eEF2K activity is inhibition of protein translation initiation, elongation, or both.

The term “eEF2K-inhibiting compound” used herein, refers to any molecule that acts with specificity to reduce eEF2K activity. In one embodiment, eEF2K-inhibiting compound reduces eEF2K activity by inhibiting its phosphorylation properties. In one embodiment, eEF2K-inhibiting compound reduces eEF2K activity by blocking its ATP hydrolysis capabilities. In one embodiment, eEF2K-inhibiting compound reduces eEF2K activity by preventing it from donating a phosphate group to an acceptor molecule. In one embodiment, eEF2K-inhibiting compound reduces eEF2K activity by reducing expression of the EEF2K gene. In one embodiment, eEF2K inhibitor is a soluble protein. In one embodiment, eEF2K inhibitor is an insoluble protein. In one embodiment, eEF2K inhibitor is a polypeptide comprising a soluble polypeptide fragment that binds to eEF2K. In one embodiment, eEF2K inhibitor is a protease. In one embodiment, eEF2K inhibitor is an antibody. In one embodiment, eEF2K inhibitor is a monoclonal antibody. In one embodiment, eEF2K inhibitor is a polypeptide comprising an antigen binding fragment of an anti-eEF2K antibody. In one embodiment, eEF2K inhibitor is a polynucleotide. In one embodiment, eEF2K inhibitor is an anti-sense polynucleotide. In one embodiment, eEF2K inhibitor is a regulatory RNA. In one embodiment, eEF2K inhibitor is a short-interfering RNA (siRNA). In one embodiment, eEF2K inhibitor is a microRNA (miRNA). In one embodiment, eEF2K inhibitor is a molecule capable of irreversible binding of eEF2K. In one embodiment, eEF2K inhibitor is a molecule capable of reversible binding of eEF2K. In one embodiment, eEF2K inhibitor is a competitive inhibitor of eEF2K. In one embodiment, eEF2K inhibitor is a non-competitive inhibitor of eEF2K. In another embodiment, eEF2K inhibitor is any small molecule capable of inhibiting eEF2K signaling.

In some embodiments, an eEF2K-inhibiting compound is capable of inducing or promoting neuron proliferation, i.e., neurogenesis. In some embodiments, an eEF2K-inhibiting compound is capable of inducing or promoting neurogenesis in an old subject's brain. As used herein, the tern “old” encompasses and subject past the embryonic neurodevelopmental period. In some embodiments, an old subject is selected from: neonatal, baby, child, adolescent, mature, and aged subject.

In some embodiments, an inhibitory molecule is a biological molecule. In some embodiments, an inhibitory molecule is a chemical molecule. In some embodiments, an inhibitory molecule is an organism-derived molecule. In some embodiments, an inhibitory molecule is a synthetic molecule. In some embodiments, an inhibitory molecule is a small molecule. In some embodiments, an inhibitory molecule is a peptide.

In some embodiments, the inhibitory molecule is an ATP-competitive inhibitor. In some embodiments, the inhibitory molecule binds to eEF2K at its ATP-binding site. In some embodiments, the inhibitory molecule is a non-ATP-competitive inhibitor. In some embodiments, the inhibitory molecule binds to eEF2K at any site but its ATP-binding site. In some embodiments, the inhibitory molecule is a substrate-competitive inhibitor. In some embodiments, the inhibitory molecule binds to eEF2K at its substrate binding site. In some embodiments, an eEF2K ATP-competitive inhibitor binds to eEF2K with greater affinity compared with a non-ATP-competitive inhibitor.

Non-limiting examples of an ATP-competitive inhibitor include, but are not limited to, Pyrroloazepines, Bis-Indoles, Aminopyrimidines, Arylindolemaleimide, Thiazoles, Paullones and Aloisines, among others.

In some embodiments, an inhibitory molecule is a Pyrroloazepine or Pyrroloazepine-derivative. In some embodiments, an inhibitory molecule is a Flavone or Flavone-derivative. In some embodiments, an inhibitory molecule is a Benzazepinoe or Benzazepinoe-derivative. In some embodiments, an inhibitory molecule is a Bis-Indole or Bis-Indole-derivative. In some embodiments, an inhibitory molecule is a Pyrrolopyrazine or Pyrrolopyrazine-derivative. In some embodiments, an inhibitory molecule is a Thiadiazolidinone or Thiadiazolidinone-derivative. In some embodiments, an inhibitory molecule is a Pyridyloxadiazole or Pyridyloxadiazole-derivative. In some embodiments, an inhibitory molecule is a Pyrazolopyridine or Pyrazolopyridine-derivative. In some embodiments, an inhibitory molecule is a Pyrazolopyridazine or Pyrazolopyridazine-derivative. In some embodiments, an inhibitory molecule is a Pyrazolopyridine or Pyrazolopyridine-derivative. In some embodiments, an inhibitory molecule is an Aminopyrimidine or Aminopyrimidine-derivative. In some embodiments, an inhibitory molecule is an Aminopyridine or Aminopyridine-derivative. In some embodiments, an inhibitory molecule is a Pyrazoloquinoxaline or Pyrazoloquinoxaline-derivative. In some embodiments, an inhibitory molecule is an Oxindole (indolinone) or Oxindole (indolinone)-derivative. In some embodiments, an inhibitory molecule is a Thiazole or Thiazole-derivative. In some embodiments, an inhibitory molecule is a Bisindolylmaleimide or Bisindolylmaleimide-derivative. In some embodiments, an inhibitory molecule is an Azaindolylmaleimide or Azaindolylmaleimide-derivative. In some embodiments, an inhibitory molecule is a Bisindolylmaleimide or Bisindolylmaleimide-derivative. In some embodiments, an inhibitory molecule is an Arylindolemaleimide or Arylindolemaleimide-derivative. In some embodiments, an inhibitory molecule is an Anilinomaleimide or Anilinomaleimide-derivative. In some embodiments, an inhibitory molecule is an Anilinoarylmaleimide or Anilinoarylmaleimide-derivative. In some embodiments, an inhibitory molecule is a Phenylaminopyrimidine or Phenylaminopyrimidine-derivative. In some embodiments, an inhibitory molecule is a Triazole or Triazole-derivative. In some embodiments, an inhibitory molecule is a Pyrrolopyrimidine or Pyrrolopyrimidine-derivative. In some embodiments, an inhibitory molecule is a Pyrazolopyrimidine or Pyrazolopyrimidine-derivative. In some embodiments, an inhibitory molecule is a Chloromethyl thienyl ketone or Chloromethyl thienyl ketone-derivative. In some embodiments, an inhibitory molecule is a heterocyclic compound. In some embodiments, an inhibitory molecule is a peptide. In some embodiments, an inhibitory molecule is a peptide-mimicking molecule. In some embodiments, an inhibitory molecule is peptidomimetics.

In some embodiments, a cell is a neuron cell. In some embodiments, a neuron is any one of: a sensory neuron, a motor neuron, an interneuron, a neurons of the brain, an astrocyte, a microglia, an ependymal cell, an oligodendrocyte, a Schwann cell, a satellite cell, an enteric glial cell, an olfactory cell, and a sheathing cell.

In some embodiments, a neuron is a neuron of the hippocampus.

In some embodiments, a neuron is a neuron of the dentate gyms (DG).

In some embodiments, a neuron is a mature excitatory neuron.

As used herein, the term “mature neuron” encompasses any terminally differentiated neuron which is no longer capable of dividing.

In some embodiments, the mature neuron comprises any neuron capable of receiving, processing, or transferring information in the central and peripheral nervous systems, or any combination thereof.

Method for identifying mature neurons are common and would be apparent to one of ordinary skill in the art. Non-limiting example for such method of identification includes, but is not limited to, immunoassays, such as immunohistochemistry using specific antibodies, e.g., anti NeuN antibody.

In some embodiments, an excitatory neuron refers to any neuron being a part of an excitatory synapse.

As used herein, the term “excitatory synapse” encompasses a set-up of a presynaptic neuron and a post synaptic neuron wherein an action potential in the presynaptic neuron may lead to a subsequent action potential in the postsynaptic cell.

In some embodiments, the excitatory neuron is releasing or secreting a neurotransmitter selected from: glutamate, acetylcholine, catecholamine, serotonin, histamine, or any combination thereof.

In some embodiments, a neuron is a neuron of a subject afflicted with a neurological disease.

As used herein, the term “neurological disease” encompasses any disease or disorder related to a component of the neural or nerve system, e.g., brain, spinal cord or other nerves. In one embodiment, neurological disease includes, but is not limited to, biochemical, electrical, or structural abnormalities, or any combination thereof, in components of the neural or nerve system.

As used herein, neurological diseases and disorder include neurodegenerative and neuromuscular diseases. None limiting examples include autonomic neuropathies, Horner syndrome, multiple system atrophy, pure autonomic failure, delirium, dementia, Alzheimer's disease, chronic traumatic encephalopathy, frontotemporal dementia, Lewy body dementia, Parkinson disease, multiple sclerosis, neuromyelitis optica, Huntington's disease, progressive supranuclear palsy, neuro-ophthalomologic and cranial nerve disorder, Isaacs Syndrome, Stiff-Person syndrome, Guillain-Barré syndrome (GBS), chronic inflammatory demyelinating polyneuropathy (CIDP), hereditary neuropathies, hereditary motor neuropathy with liability to pressure palsies (HNPP), amyotrophic lateral sclerosis (ALS) and other motor neuron diseases (MNDs), myasthenia gravis, nerve root disorders, herniated nucleus pulposus, peripheral neuropathy, mononeuropathies, multiple mononeuropathy, polyneuropathy, brachial plexus and lumbosacral plexus disorders, spinal muscular atrophies (SMAs), thoracic outlet compression syndromes (TOS), Creutzfeldt-Jakob Disease (CJD), Gerstmann-Sträussler-Scheinker Disease (GSS), seizure disorders, spinal cord disorders, stroke, depression, epilepsy, memory loss, and cognitive impairment.

As used herein “neurodevelopmental disease or disorder” is a neurologically based condition that appears early in childhood, typically before school entry. Such disorders impair development of personal, social, academic, and/or occupational functioning and typically involve difficulties with the acquisition, retention, or application of specific skills or sets of information. The disorders may involve dysfunction in attention, memory, perception, language, problem-solving, or social interaction. Non-limiting examples of neurodevelopmental diseases or disorders include, learning disability, attention-deficit/hyperactivity disorder (ADD/ADHD), autism spectrum disorders, and intellectual disability.

As used herein “neural cancer disease” is a disease associated with neural cell proliferation. Non-limiting types of neural cancer include acoustic neuroma, astrocytoma, chordoma, CNS lymphoma, craniopharyngioma, glioma, medulloblastoma, meningioma, metastatic brain tumor, primary brain lymphoma, spinal cord tumor, oligodendroglioma, pituitary tumor, primitive neuroectodermal tumor, Schwannoma, juvenile pilocytic astrocytoma, pineal tumor and rhabdoid tumor. In one embodiment, astrocytoma refers to tumor derived from astrocytes including but not limited to grade I—pilocytic astrocytoma, grade II—low-grade astrocytoma, grade III—anaplastic astrocytoma and grade IV—glioblastoma. In one embodiment, other types of glioma include but not limited to brain stem glioma, ependymoma, mixed glioma, optic nerve glioma and subependymoma.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, and pig. In one embodiment, the subject is a human. Human subjects may also include fetuses. In one embodiment, a subject in need thereof is a subject afflicted with and/or at risk of being afflicted with a condition associated with neural disease or disorder. In one embodiment, a subject in need thereof is a subject afflicted with and/or at risk of being afflicted with a condition associated with increased neural cell proliferation.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

Method of Screening

A method of screening for a compound suitable for treating a neurological disease, the method comprising contacting a neuron with a compound, and measuring activity of eEF2K in the presence of the compound, wherein reduction of eEF2K activity in the neuron in the presence of the compound compared to eEF2K activity in neuron in the absence of the compound is indicative that said compound is suitable for treating a neurological disease.

In another embodiment described herein, the present invention provides a method of screening for novel eEF2K-inhibiting compounds suitable for treating neurological diseases.

Assays for identification of novel eEF2K-inhibiting compounds are well known to one skilled in the art and include but are not limited to preparation and screening of chemical combinatorial libraries. Such combinatorial chemical libraries include, but are not limited to, peptide libraries (see, e.g., U.S. Pat. No. 5,010,175, Furka, (1991) Int. J. Pept. Prot. Res. 37: 487-493, Houghton, et al. (1991) Nature 354: 84-88). Peptide synthesis is by no means the only approach envisioned. Other chemistries for generating chemical diversity libraries can also be used. Such chemistries include, but are not limited to; peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs, et al. (1993) Proc. Nat'l Acad. Sci. USA 90: 6909-6913), vinylogous polypeptides (Hagihara, et al. (1992) J. Amer. Chem. Soc. 114: 6568), nonpeptidal peptidomimetics with a β-D-Glucose scaffolding (Hirschmann, et al., (1992) J Amer. Chem. Soc. 114: 9217-9218), analogous organic syntheses of small compound libraries (Chen, et al. (1994) J. Amer. Chem. Soc. 116: 2661), oligocarbamates (Cho, et al, (1993) Science 261:1303), and/or peptidyl phosphonates (Campbell, et al, (1994) J. Org. Chem. 59: 658; Gordon, et al., (1994) J. Med. Chem. 37: 1385), nucleic acid libraries (see, e.g., Strategene, Corp.), peptide nucleic acid libraries (see, e.g., U.S. Pat. No. 5,539,083) antibody libraries (see, e.g., Vaughn, et al. (1996) Nature Biotechnology 14(3): 309-314), and PCT/US96/10287), carbohydrate libraries (see, e.g., Liang, et al. (1996) Science 274:1520-1522, and U.S. Pat. No. 5,593,853), and small organic molecule libraries (see, e.g., benzodiazepines: Baum (1993) C&EN, January 18, page 33; isoprenoids: U.S. Pat. No. 5,569,588; thiazolidinones and metathiazanones: U.S. Pat. No. 5,549,974; pyrrolidines: U.S. Pat. Nos. 5,525,735 and 5,519,134; morpholino compounds: U.S. Pat. No. 5,506,337; benzodiazepines: U.S. Pat. No. 5,288,514; and the like).

In some embodiments, after a library has been created, the compounds are screened for eEF2K kinase inhibitory activity. In some embodiments, the compounds are screened for binding to eEF2K, such as within the ATP-binding pocket. In some embodiments, the compounds are screened for eEF2K ATP-competitive activity. In some embodiments, the compounds are screened for binding to eEF2K, such as at any site but the ATP-binding pocket, including, but not limited to the binding site of the phosphorylation substrate. In some embodiment, the compounds are screened for eEF2K non-ATP-competitive activity.

In some embodiments, the inhibitory effect of an assayed compound over eEF2K is assessed in vivo. In some embodiments, the inhibitory effect of an assayed compound over eEF2K is assessed in vitro.

Neuronal cell lines which are common and many varieties of which can be used to screen for activity, would be apparent to one of ordinary skill in the art.

As used herein, the terms “subject” or “individual” or “animal” or “patient” or “mammal,” refers to any subject, particularly a mammalian subject, for whom therapy is desired, for example, a human.

Composition

According to some embodiments, there is provided a composition comprising an eEF2K-inhibiting compound and an acceptable carrier.

According to some embodiments, the composition is a pharmaceutical composition. In some embodiments, the composition comprises a pharmaceutically acceptable carrier, excipient, or adjuvant, is provided.

In some embodiments, the composition comprises a therapeutically effective amount of the eEF2K-inhibitor. In some embodiments, there is provided a pharmaceutical composition comprising an eEF2K-inhibitor or an eEF2K-inhibiting compound, for use in treatment of a neurological disease.

As used herein, the term “carrier”, “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents that may be useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The carrier may comprise, in total, from about 0.1% to about 99.99999% by weight of the pharmaceutical compositions presented herein.

As used herein, the term “therapeutically effective amount” refers to a concentration of a eEF2K-inhibitor effective to treat or prevent a disease or disorder in a subject, such as a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising,” “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference. Other general references are provided throughout this document.

Materials and Methods

Mice

eEF2K-KO mice (eEF2K −/−), in which coding exons 7, 8, 9, and 10 of eEF2K were deleted, were generated. The inventors derived eEF2K wild-type (WT) and KO (eEF2K −/−) littermates by crossing heterozygous mice (eEF2K +/−). eEF2K foxed mice are homozygous to the floxed eEF2K gene. Conditional eEF2K-KO mice were generated by viral injection of AAV-CaMKII Cre to eEF2K foxed mice. eEF2K foxed mice were generated by the laboratory of Christopher G. Proud. C57BL/6 WT mice were obtained from Envigo RMS, Jerusalem, Israel, and after a week of acclimation to the facility were used for experiments. The eIF2α-KI and their WT littermate mice were used for BrdU and DCX analysis. These mice were obtained from Jackson laboratory, stock number: 017601.

The mice used in this study were 2 and 14 months of age, and all experiments were done during the light phase (7 AM-7 PM). All procedures were approved by the University of Haifa Animal Care and Use committee and were in accordance with the National Institutes of Health guidelines for ethical treatment of animals.

Surgery and Virus Injection

For all surgeries, naive young and old eEF2K floxed mice received norocarp (10 mg/kg body weight) prior to surgery and 24 h later to minimize pain. Mice were anesthetized using isoflurane 3%. Mice were placed in a stereotaxic apparatus (KOPF, 1900) and maintained on isoflurane flow of 1.5% at the time of the surgery. Two holes (0.4 mm) were drilled in both hemispheres and 0.5 μl virus was injected bilaterally into the dorsal dentate gyms using a 10 μl Hamilton syringe (A-P: −2.0 mm; D-V: 1.9 mm; M-L: ±1.3 mm). The needle was left in the injection site for 10 min to ensure sufficient distribution of the virus and minimize retraction, and the skin was closed using 3M Vetbond glue. CaMKII-GFP/Cre-injected mice were used 6-7 weeks post-infection for behavioral, neurogenesis, and biochemical analysis. The viruses were purchased from Penn Vector Core, University of Pennsylvania. Viruses used in this study: AAV1.CaMKII0.4.eGFP.WPRE.rBG; AAV1.CaMKII.HI.eGFP-Cre. WPRE.SV40; AAV1.hSyn.eGFP.WPRE.bGH; and AAV1.hSyn.H1.eGFP-Cre.WPRE. SV40

Steady-State Deep Proteomics

Sample Preparation

All chemicals were purchased from Sigma Aldrich, unless stated otherwise. Tissues were mechanically homogenized in the presence of lysis buffer (5% SDS in 50 mM Tris-HCl, pH 7.4). Protein concentration was measured using the BCA assay (Thermo Scientific, USA). Samples containing 100 μg of total protein were reduced with 5 mM dithiothreitol and alkylated with 10 mM iodoacetamide in the dark. Each sample was loaded onto S-Trap mini-columns (Protifi, USA) according to manufacturer's instructions. In brief, after loading, samples were washed with 90:10% methanol/50 mM ammonium bicarbonate. Samples were then digested with trypsin (1:50 trypsin/protein) for 1.5 h at 47° C. The digested peptides were eluted using 50 mM ammonium bicarbonate; trypsin was added to this fraction and incubated overnight at 37° C. Two more elutions were made using 0.2% formic acid and 0.2% formic acid in 50% acetonitrile. The three elutions were pooled together and vacuum-centrifuged to dryness. Samples were kept at −80° C. until analysis.

Liquid Chromatography

ULC/MS grade solvents were used for all chromatographic steps. Each sample was fractionated offline using high pH reversed phase followed by online low pH reversed phase separation. Samples containing 100 μg digested protein were loaded and separated into fractions using High Performance Liquid Chromatography (Agilent 1260 uHPLC). Mobile phase contained: A) 20 mM ammonium formate pH 10.0, B) acetonitrile. Peptides were separated on an) (Bridge BEH C18 column (2.5 μm, 3×100 mm, Waters) using the following gradient: 3% B for 2 minutes, linear gradient to 40% B in 50 min, 5 min to 95% B, maintained at 95% B for 5 min and then back to initial conditions. Peptides were fractionated into 15 fractions. Since low pH fractionation is not perfectly orthogonal to the downstream high pH reversed phase separation, the fractions were then pooled as follows: 1 with 8, 2 with 9, 3 with 10, 4 with 11, 5 with 12, 6 with 13, and 7 with 14-15, in order to achieve a balanced chromatogram for each pooled fraction and obtain maximal peak capacity. Each fraction was vacuum centrifuged to dryness, then reconstituted in 25 μL in 97:3 acetonitrile: water+0.1% formic acid. Each pooled fraction was then loaded and analyzed using split-less nano-Ultra Performance Liquid Chromatography (10 kpsi nanoAcquity; Waters, Milford, Mass., USA). The mobile phase contained: A) H₂O+0.1% formic acid and B) acetonitrile +0.1% formic acid. Desalting of the samples was performed online using a Symmetry C18 reversed-phase trapping column (180 μm internal diameter, 20 mm length, 5 μm particle size; Waters). The peptides were then separated using a T3 HSS nano-column (75 μm internal diameter, 250 mm length, 1.8 μm particle size; Waters) at 0.35 μL/min. Peptides were eluted from the column into the mass spectrometer using the following gradient: 4% to 30% B in 105 min, 30% to 90% B in 5 min, maintained at 90% for 5 min and then back to initial conditions.

Mass Spectrometry

The nanoUPLC was coupled online through a nanoESl emitter (10 μm tip; New Objective; Woburn, Mass., USA) to a quadrupole orbitrap mass spectrometer (Q Exactive Plus, Thermo Scientific) using a FlexIon nanospray apparatus (Proxeon). Data were acquired in DDA mode, using a Top10 method. MS1 resolution was set to 70,000 (at 400 m/z) and maximum injection time was set to 60 msec. MS2 resolution was set to 17,500 and maximum injection time of 60 msec.

Data Analysis

Raw data were processed with MaxQuant v1.6.0.16. The data were searched with the Andromeda search engine against the mouse (Mus musculus) protein database as downloaded from Uniprot (www.uniprot.com) and appended with common lab protein contaminants. Enzyme specificity was set to trypsin and up to two missed cleavages were allowed. Fixed modification was set to carbamidomethylation of cysteines and variable modifications were set to oxidation of methionines, and deamidation of glutamines and asparagines. Peptide precursor ions were searched with a maximum mass deviation of 4.5 ppm and fragment ions with a maximum mass deviation of 20 ppm. Peptide and protein identifications were filtered at an FDR of 1% using the decoy database strategy (MaxQuant's “Revert” module). The minimal peptide length was 7 amino-acids and the minimum Andromeda score for modified peptides was 40. Peptide identifications were propagated across samples using the match-between-runs option checked. Searches were performed with the label-free quantification option selected. The quantitative comparisons were calculated using Perseus v1.6.0.7. Decoy hits were filtered out, as well as proteins that were identified on the basis of one peptide only, and only proteins that had at least two valid values in at least one experimental group were kept. A Student's t-test, after logarithmic transformation, was used to identify significant differences across the biological replica. Fold changes were calculated based on the ratio of geometric means of the WT versus eEF2K-KO experimental groups.

Statistical Analysis

To identify robust significant changes in the abundance of specific proteins, the inventors determined ±15% and ±50% cutoffs of significant change. This analysis of 354 proteins identified by MaxQuant software revealed 190 and 37 proteins that significantly passed the ±15% and ±50% cutoffs, respectively. Decorin and vimentin (neurogenesis-related proteins) were used as targets to validate the proteomics data. Vimentin passed the 15% cutoff of change and decorin passed the 50% cutoff of change. Enrichment analysis of biological processes was performed using Panther Classification System. Significance of enrichment was concluded when P<0.05 by Fisher's exact test. Heat map was done utilizing Heatmapper software.

Dentate Gyrus Tissue Preparation and Western Blotting

Mouse brains were quickly excised and snap frozen immediately in liquid nitrogen, and then transferred to −80° C. for later use. The brains were kept in the cryostat cabin for 15 min at −15° C. before starting tissue punching. Four 500 μm thick coronal sections were sliced slowly, and the DG and cortex tissues from both hemispheres were collected by a mouse tissue puncher. The DG and cortex tissues were transferred to a new tubes and kept immediately on dry ice and then transferred to −80° C. until further use. Brain tissues were homogenized in 100 μl of ice-cold homogenization buffer (HEPES 10 mM pH 7.4, EDTA 2 mM pH 7.4, EGTA 2 mM pH 7.4, DTT 0.5 mM (all from Sigma-Aldrich, Rehovot, Israel), 1× protease inhibitor mixture (Sigma-Aldrich Rehovot, Israel); and 1× phosphatase inhibitor mixture (Sigma-Aldrich)). Protein concentration was determined by a BCA kit (CYANAGEN, PRTD1, 0500). Protein Samples were prepared in SDS sample buffer (22.22% glycerol, 22.22% SDS (20% stock), 26.66% (0.5 M Tris PH 6.8), rest DDW) and β-Mercaptoethanol, then were boiled in 100° C. for 5 min. Each sample was divided to 3 aliquots. Each aliquot was thawed on ice and used for blotting only once or twice. The samples were subjected to 10% or 12% gel SDS-PAGE (electrophoresed on Bio-Rad PAGE apparatus) and Western blot analysis. Each sample was loaded with the same amount of total protein (8-12 μg; according to antibody linearity). After transfer to a 0.2 mm pore size nitrocellulose membrane or PVDF membrane, the blots were blocked with 5% bovine serum albumin (BSA) or non-fat dry milk (blotting-grade blocker, Bio-Rad) in Tris-buffered saline plus 0.5% tween-20 (TBST) at room temperature for 1 hr. The blots were incubated overnight with the suitable primary antibodies. The blots were then subjected to three 5 min washing steps in TBST, after which they were incubated with the corresponding HRP-conjugated secondary antibodies for 1 h at room temperature followed by three 10 min washing steps with TBST. Immunodetection was performed with the enhanced-chemiluminescence EZ-ECL kit (Biological Industries, Israel). The immunoblots were quantified with a CCD camera and Quantity One software (Bio-Rad).

Western Blot Analysis

For all tested proteins, polyacrylamide gels were freshly prepared and used on the same day. Polyacrylamide gels (10%, Tris pH 8.8) were used to analyze the expression of eEF2, pThr56eEF2, vimentin, decorin, AMPK, pThr172AMPK, KIFSA, ERK2, pERK2, pS6K1, S6K1 and tubulin. In addition, criterion gels (BIO-RAD) were used to analyze the expression of NSF protein in the different fractions. Samples containing 10 μg protein were loaded for the detection of each protein. Samples containing 8 μg protein were loaded for ERK and pERK and 12 protein for S6K1 and pS6K1. Each immunoblot was measured relative to the background and normalized to the endogenous control (tubulin), taken from the same gel. Mature-BDNF was analyzed using 12% (Tris pH 8.8) polyacrylamide gel and was measured relative to the background and normalized to tubulin from the same gel. Samples containing 12 μg protein were loaded for mature-BDNF. The phoshpo-protein was normalized to tubulin taken from the same gel as well. Ratios of phospho-protein/protein levels and protein/tubulin (housekeeping gene) are shown in the figures.

Immunohistochemistry and Quantification

Adult mice were first anesthetized with isoflurane in an anesthesia chamber. After the mice were fully anesthetized, they were perfused transcardially with cold PBS followed by 4% paraformaldehyde (PFA) in 0.1 M PBS (pH 7.4). The brains were dissected immediately and post-fixed overnight at 4° C. in 4% PFA. Afterwards, the brains were cryoprotected in 30% sucrose in 0.1 M PBS (pH 7.4) for 48 h at 4° C. and then transferred to −80° C. Coronal sections (30 μm thickness) were obtained (bregma 1.54 mm to −2.92 mm) using a Leica cryostat (CM 1950) in six matched sets and stored in 0.1 M PBS (pH 7.4) with 0.01% sodium azide at 4° C. Immunohistochemistry was performed on one set of slices.

Adult-Neurogenesis Analysis

Modified protocols for BrdU and DCX staining were used.

BrdU Staining

BrdU (Sigma) solution was prepared at a concentration of 20 mg/ml in 0.9% NaCl. The final dose was 200 mg/kg, injected intraperitoneally twice at a dose of 100 mg/kg in a 2 h interval. The mice were perfused 24 h following the last BrdU injection in order to examine proliferation changes. Sections were obtained as above and stored in PBS containing 0.01% sodium azide at 4° C. For immunolabeling, the sections were washed three times with PBS. Then, the DNA denaturation was performed by incubating the sections in HCl 2 M for 15 min at 37° C. Afterwards, the sections were washed with PBS and incubated with blocking buffer (10% FBS, 0.3% BSA in 0.3% Triton X-100-PBS) for 2 h. The sections were incubated overnight with BrdU primary antibody (1:200). On the second day, the sections were left on the shaker at RT for 1 h, and then washed with 0.3% Triton X-100-PBS three times. One set of sections was used for DAB staining and the other was used for immunofluorescence staining. For DAB staining, the sections were incubated with the first secondary antibody, biotinylated anti-Rat 1:500 for 1 h. Then, the sections were washed and re-incubated with the second antibody, horse radish peroxidase 1:500, for 1 h. The sections were washed with 0.3% Triton X-100-PBS and incubated in DAB solution for 5 min. After incubation in the DAB solution, the sections were washed with PBS and mounted onto superfrost slides. The quantification of BrdU positive cells in the DG was done using bright field and a 40× objective. For BrdU immunofluorescence staining, on the second day, the sections were shaken with the primary antibody for 1 h in RT. Then the sections were washed with 0.3% Triton-X-100-PBS and incubated with the fluorescent-label-coupled secondary antibody, 1:500, for 1 h. Finally, the sections were washed and mounted onto Superfrost slides. Mounting solution contained DAPI (Vector) was added before adding the coverslips. Slides were visualized and acquired using inverted system microscope, Olympus 1X81 (Olympus CellSens Dimension). The quantification of BrdU positive cells was done under 40× magnification.

DCX Staining

Sections were stored in PBS containing 0.01% sodium azide at 4° C. One set of sections was used. On the first day, sections were washed with 0.3% Triton X-100-PBS and incubated with blocking buffer (10% FBS, 0.3% BSA in 0.3% Triton X-100-PBS) for 2 h. Then, the sections were incubated with primary antibody, 1:1,000, overnight in 4° C. On the second day, the sections were shaken with the primary antibody for 1 h in RT. Afterwards, the sections were washed with 0.3% Triton X-100-PBS and incubated with fluorescent-label-coupled secondary antibody, 1:500, for 1 h. Finally, the sections were washed with PBS and mounted onto SuperFrost slides. Mounting solution contained DAPI (Vector) was added before adding the coverslips. Slides were visualized and acquired using a vertical light microscope (Olympus cellSens Dimension) at 20× magnification and inverted system microscope, Olympus 1X81 (Olympus cellSens Dimension) at 40× magnification. Images were processed using Image-J. The quantification of DCX positive neurons was done in the DG using a 20× objective. For BrdU and DCX analysis, positive cells in the granule cell layer and subgranular zone were counted manually along the dorsal to ventral axis of the DG (1 set of 6). Summing the counted cells and multiplying by 6 yielded the total cell count per mouse.

Antibodies

Antibodies Used in this Study:

Primary Antibodies:

eEF2 antibody (1:10,000; ab40812, Abcam), pThr56eEF2 antibody (1:10,000; ab53114, Abcam), Proxy1 antibody (1:1,000; ab38692, Abcam), Decorin antibody (1:3,000; ab175404, Abcam), Vimentin antibody (1:1,000; ab20346, Abcam), Tubulin antibody (1:40,000; SAB4500087, Sigma), BrdU (1:200; ab6326, Abcam), DCX (1:1,000; ab18723, Abcam) , BDNF (1:500,SC-546 (N-20), Santa Cruz), AMPK (1:1,000, 2532, Cell signaling Technology), pT172AMPK (1:1,000, 2535, Cell signaling Technology), ERK (1:3,000, 4695S, Cell signaling Technology), pERK (1:2,000, 4370L, Cell signaling Technology), S6K1 (1:1,000, 9202S, Cell signaling Technology), pT389S6K1 (1:750, 9205S, Cell signaling Technology), KIF5A (1:1,000, NB-120-5628, NOVUS BIOLOGICALS), NSF (1:2,000, #2145S, Cell signaling Technology).

Secondary Antibodies:

Donkey anti-rabbit ((Alexa Fluro 568), 1:500, ab175470, Abcam), Goat anti-Rat ((Alexa Fluro 568), 1:500, ab175476, Abcam), Streptavidin horseradish peroxidase (HRP) conjugate (1:500; 43-4323, Invitrogen), Biotinylated anti-rat IgG (1:500; BA-9401, Vector), Peroxidase-conjugated affiniPure goat anti-rabbit (1:10,000; 111-035-144, Jackson), Peroxidase-conjugated AffiniPure goat anti-mouse (1:10,000; 115-035-062, Jackson).

Behavioral Paradigms

Context Discrimination of Fear Conditioning

A modified protocol of the context discrimination paradigm was used. For three days, mice received one trial of the following: 180 s pre-shock followed by a 2 s foot shock of 0.4 mA, and then the mice were taken out 60 s after termination of the shock (total trial was 242 s). Freezing levels were quantified in the initial 180 s prior to the shock. The first test was done on day 4, in which the animals were exposed to the training context (Context A; without delivering shock) or a highly similar context (Context B), a less similar context (context C), and a different context (context D) for 242 sec. Following 14 and 28 days, the mice were exposed to Context A and similar contexts in counterbalanced design. The experiments were done in Coulburn Habitest fear conditioning chambers. Two days before starting the experiments, mice were brought out of the vivarium and allowed to habituate for 4 h in a different room. Context A (the training context), stainless-steel bars were exposed, the fan and light were on, the letter A in black was added to the back wall, and a cleaning detergent of “Sano” was added as an olfactory cue. For context B, a black board was added instead of the letter A and diluted cleaning detergent was added as an olfactory cue. For context C, a white sheet of paper was added to one of the chamber walls the black board was kept as in context B, ethanol 50% was added as an olfactory cue, and the room was illuminated with dim light. For context D, the mice were exposed to a novel context in which the stainless-steel bars were covered by a black plastic board, two black circles were added to the walls, a black and white pattern square was added to one of the walls, and the room was illuminated with dim red light. Freezeframe and Freezeview (Actimetrics software version 3, Coulbourn instruments) were used for recording and analyzing freezing behavior. Context discrimination index was calculated as: (% freezing in context A-% freezing in context B)/(Total % of freezing in contexts A and B).

Enriched Environment Protocol and TMZ i.p. Injection

C57BL/6 WT mice were divided into four groups: 1. Controls, without enrichment, treated with vehicle; 2. Mice in an enriched environment, treated with vehicle; 3. Mice in an enriched environment, treated with DNA alkylating agent Temozolomide (TMZ, Sigma); and 4. Mice in a standard (non-enriched) environment, treated with TMZ.

The enriched environment (groups 2 and 3) included a running wheel, two different objects, small ball, a short plastic tube, bedding, and a nest. Control animals (groups 1 and 4) were in standard cages including bedding and nest. In both conditions, three mice were housed per cage for four weeks. The mice received a TMZ/vehicle i.p. injection three times a week. On day 29, the mice received a BrdU i.p. injection and were perfused 24 h later for IHC analysis. For neurogenesis suppression, TMZ was dissolved in DMSO at 25 mg/ml and diluted to 1.25 mg/ml in sterile saline. The mice were given i.p. injections of saline +5% DMSO (vehicle) or TMZ on three consecutive days followed by four days with no injection for four weeks. On the last week, BrdU was given i.p. in 0.9% NaCl at 200 mg/kg body weight. The mice maintained normal body weight throughout the experiment.

For biochemical analysis of pThr56eEF2/eEF2 levels following enriched environment treatment, the same conditions as above were used. Two groups of mice were used. Control mice were in standard cages, whereas enriched environment mice were in an enriched environment. The mice were sacrificed 4 weeks after the treatment and the DG lysates were analyzed for biochemical changes.

TMZ i.p. Injection in CaMKII-Cre and CaMKII-GFP Mice

Sixteen (16) eEF2K double foxed mice were injected with CaMKII-Cre and GFP viruses to the DG and 3 weeks following virus injection were injected i.p TMZ and vehicle. TMZ was dissolved in DMSO at 25 mg/ml and diluted to 1.25 mg/ml in sterile saline. The mice were given i.p. injections of saline +5% DMSO (vehicle) or TMZ on three consecutive days followed by four days with no injection for four weeks. On the last week, BrdU was given i.p. in 0.9% NaCl at 200 mg/kg body weight. The mice maintained normal body weight throughout the experiment. Four groups of mice (CaMKII-GFP/V, CaMKII-Cre/V, CaMKII-GFP/TMZ, CaMKII-Cre/TMZ) were perfused for IHC analysis of BrdU and DCX.

Social Interaction Behavior

The mice were habituated in a 3-chamber arena for 10 min on day 1. On day 2, the mice underwent the social interaction and social novelty tests. The arena had thee chambers: left, right, and center. In the left and right chambers, the stranger mouse (social stimulus) and the empty wire cage were placed, whereas the center chamber was used to release the animal. For the social interaction test, the mouse was released in the center chamber and immediately the doors leading to the stranger and the empty wire cage were opened simultaneously. The trial was held for 20 min and the stranger/wire cage places were counterbalanced. The time spent exploring the stranger and the empty wire cage was recorded using EthoVision XT (Noldus). Sociability index was calculated as stranger mouse exploration out of total exploration of both stranger and empty wire cage. After an interval of 5 min, the social novelty interaction test was performed. The empty wire cage was replaced with a novel mouse, and the previous mouse (familiar) was kept in this test. The trial was held for 20 min, and the mouse was allowed to explore the familiar and the novel mouse at the same time. Both mice were counterbalanced between the chambers. Social novelty exploration index was calculated as novel mouse exploration out of total exploration of both mice. The recording was done using EthoVision XT software.

Forced Swim Test

WT mice were injected with ketamine 5 mg/kg or saline i.p., and 30 min later together with the CaMKII Cre/GFP-injected mice were all subjected to the forced swim test (FST). The mice were placed in a 5-liter transparent glass beaker containing 4-liter warm water at 24° C. The mice were video recorded for 6 min. The last 4 min were manually scored for immobility. Total immobility time (in seconds) is presented in the results.

Open Field Test

The mice were habituated in a square arena 50×50 cm (Noldus Information Technology, Canada). The mice were allowed to explore the arena for 10 min. At the beginning of the trial, the mice were placed in the center of the arena, and the arena was always cleaned with 50% ethanol between mice. After the exploration, the mice were returned to their home cage. Animal behavior was recorded and analyzed using EthoVision XT9 software.

Novel Object Recognition

The apparatus consisted of a square arena of 50×50 cm (Noldus Information Technology, Canada). Two objects were placed in a symmetrical position about 6 cm from the walls. Mice were habituated to the empty arena for 1 day by exploring the arena for 10 min. After exploration, the mice were returned to the home cage. After the habituation period, the mice underwent acquisition for 2 similar objects for 10 min and given 2 trials per day for 2 days. After the acquisition period, mice were tested for novel object recognition test, in which one of the objects was replaced by new object which has different shape and color. The mice were allowed to explore the arena for 10 min. At the beginning of each trial, the mice were placed at the center of the arena. The arena was always cleaned using 50% ethanol between trials to avoid odor effects. Exploration of the objects was defined as directing the nose or touching the object at a distance of 2 cm. Climbing the object was not considered to be exploration. Animal behavior was recorded, and the data were analyzed by EthoVision XT9 software (Noldus Information Technology, Canada).

Morris Water Maze

The Morris water maze (MWM) consisted of a black circular pool (120 cm; 50 cm) filled with warm water 22° C. mixed with milk powder. Distal cues were used in the divided 4 quadrants. The mice learned to use distal cues to navigate a direct path to the hidden platform. A weak protocol of MWM was used. During the learning period, the mice were given one trial per day for 5 days. After 3 and 5 days of learning, the first probe test (PT1) and the second probe test (PT2) were performed respectively under full-cues. In both probe tests, the platform was removed from the pool. During the learning period, escape latency was measured in each trial. Percentage of time spent in the platform quadrant in the probe test was measured, and the data were analyzed by EthoVision XT9 software.

Reversal Morris Water Maze

Two days following the second probe test (PT2) of the MWM paradigm, reversal MWM test was performed. In this test, the platform was relocated in the opposite quadrant, and one trial per day was performed. Each trial lasted for 1 min, escape latency was measured, and previous platform crossings were analyzed. The data were analyzed by EthoVision XT9 software.

One-Cue Test of Morris Water Maze

Two weeks after the last probe test (PT2), one cue test was performed, in which three cues were removed from the pool, and one cue located more distally from the platform was kept. Escape latency to the hidden platform was measured. Full cue test was performed following the one cue test. Latency to platform was measured. The data were analyzed by EthoVision XT9 software.

LTP Protocol

Adult male eEF2K double floxed mice virus transfected with CaMKII GFP and CaMKII Cre vectors were anesthetized with urethane (injected i.p. 1.2 g/kg), which was supplemented throughout surgery and recording as required. Mice were placed in a stereotaxic frame and body temperature was maintained at 37° C. In one hemisphere only, a bipolar stimulation electrode (NE-200, 0.5 mm tip separation, Rhodes Medical Instruments, Wood hills, Calif.) was positioned ipsilaterally onto the perforant path (3.8 mm posterior to bregma, 2.7 mm lateral to midline, and 1.5 mm from the brain surface). Insulated tungsten recording electrode (0.075 mm; A-M Systems) was positioned in the hilus of the dentate gyms (2 mm caudal to bregma, 1.5 mm lateral to the midline, and 1.5-1.8 mm from the brain surface). Electrodes were lowered into the brain in 0.1 mm increments while monitoring the laminar profile of the response waveform evoked by a 300 μA test pulse stimulus applied. Electrode positioning was limited to three penetrations while maximizing the field extracellular postsynaptic potential (fEPSP) response. To generate input/output (I/O) curves, five stimulus intensities ranging from 60 μA to 300 μA were applied in a randomized sequence. The maximal slope of the initial rising phase of the fEPSP was measured. After generating an I/O curve, a stable 20 min baseline of evoked potentials was recorded (pulse-width 0.1 ms, at 0.033 Hz) before HFS. HFS was delivered at an intensity that produced a population spike 30% of the maximum. LTP was induced either single train (Weak HFS) or four trains (Strong HFS) of stimuli applied with an interval of 10 sec; each train had 15 pulses at 200 Hz (pulse-width 0.1 ms;). The stimulus intensity used for HFS was twice that used for test pulses. Evoked responses were recorded for 180 min post-HFS. Changes in the fEPSP slope were expressed in as percent of baseline (20 min preceding HFS). After recordings were completed, the electrodes were removed, the animal was sacrificed, and the dentate gyri were micro-dissected and immediately frozen on dry ice for later use.

Statistical Analysis

Statistical analysis was done with IBM SPSS statistics 21 and GraphPad Prism 8. Graphs were generated with Microsoft Excel worksheet and GraphPad Prism. Normal distribution (Kolmogorov-Smirnova and Shapiro-Wilk tests) and approved homogeneity tests were analyzed. For normally distributed data, differences among multiple groups were assessed by one-way, two-way, and repeated-measures analysis of variance. Post-hoc differences were determined by Tukey's test and independent sample t-test, when significant main effects or interactions were detected. For independent samples, two tailed t test was conducted when two groups were compared. For non-parametric tests, Friedman test, Kruskal-Wallis test, and Mann-Whitney U test were conducted when the data were non-normally distributed. Data are presented as mean ±SEM. The accepted value of significance for all tests was set at p<0.05.

### RESULTS

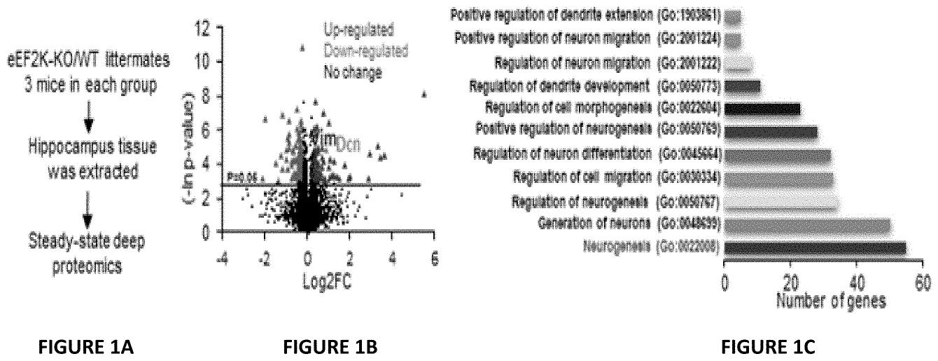

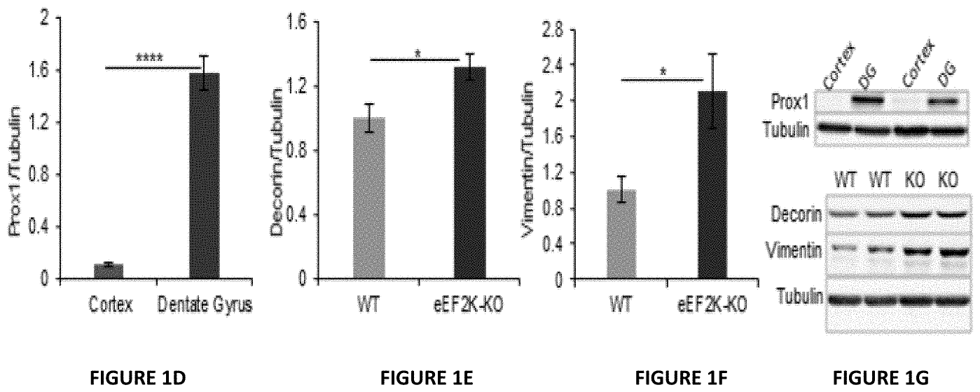

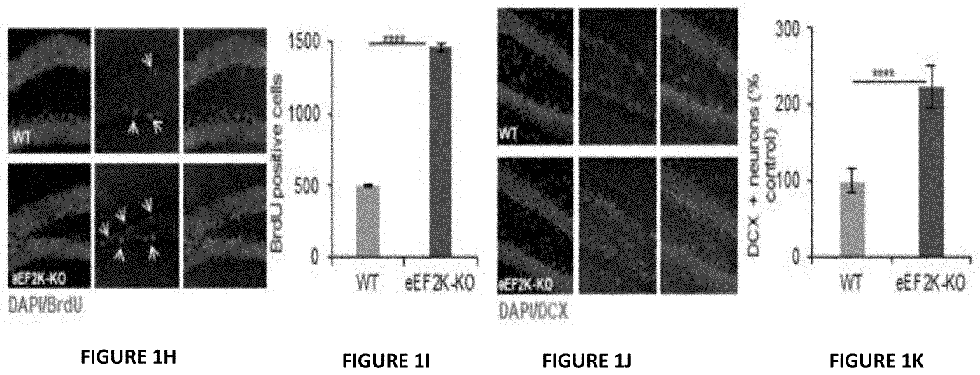

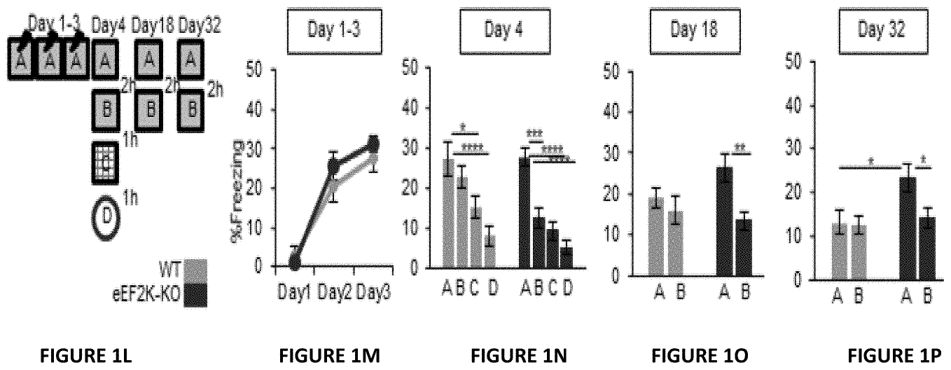

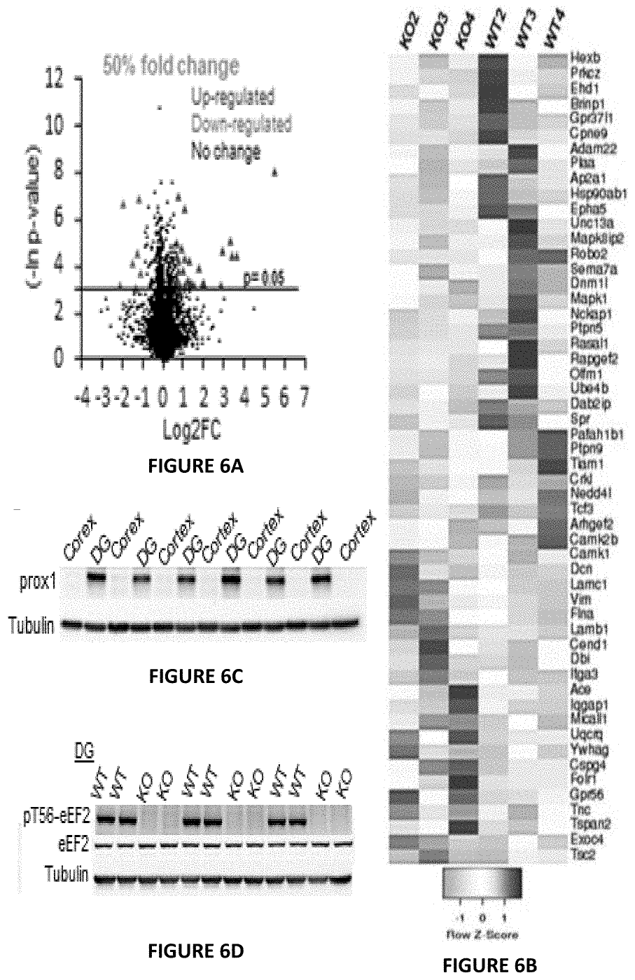

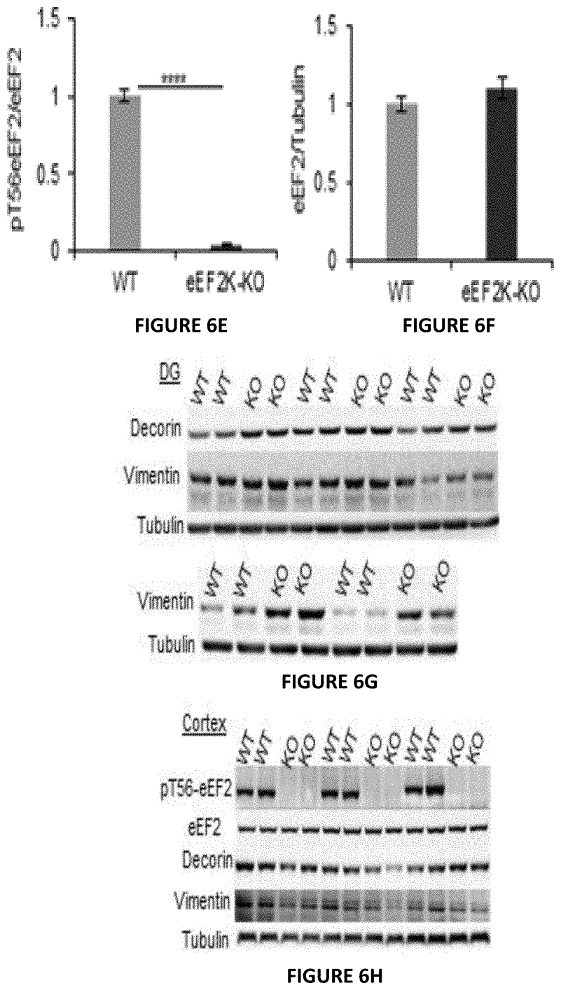

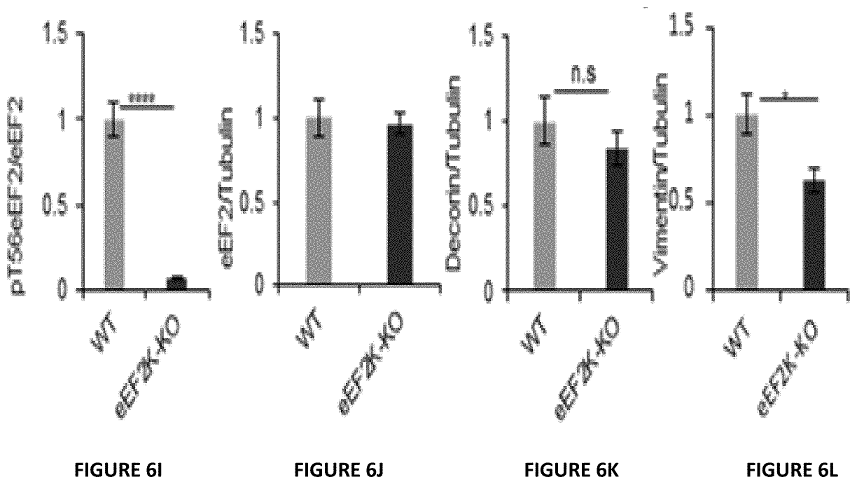

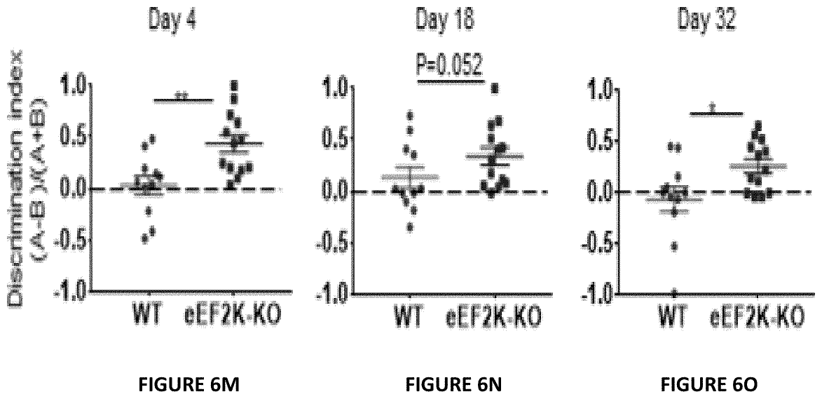

In order to measure the effect of turning on translation elongation via the eEF2 pathway in the hippocampus, the inventors first determined the effect of eEF2 dephosphorylation on proteostasis in the mature brain. Steady-state deep proteome analysis of hippocampus tissue from eEF2K-KO mice and WT littermates identified 6,265 proteins in total. Expression levels of 354 proteins were significantly (p<0.05) different between eEF2K-KO and WT mice with no peEF2 in the hippocampus, as expected (dentate gyms, DG FIG. 1A) or cortex of eEF2K-KO (FIGS. 6D-6F, and 6H-6J). Enrichment analysis of biological processes for all 354 proteins identified 54 significantly enriched neurogenesis-related proteins and others involved in neuronal differentiation, development, migration, and morphogenesis (FIGS. 1B-1C, and FIGS. 6A-6B). Validation WB analysis corroborated proxy1 (DG marker) enrichment in the DG compared to the cortex in WT mice (T_{5.120}=−10.734, Cortex versus Dentate gyms, P=0.00000015, Cortex: n=7, DG: n=6; FIGS. 1D-1G, and FIG. 6C) and confirmed increased levels of two key proteins involved in neurogenesis, decorin and vimentin (T₁₀=−2.678, WT versus eEF2K-KO, P=0.023, WT: n=6, eEF2K-KO: n=6; FIGS. 1E and 1G, FIG. 6G, T_{11.129}=−2.511, WT versus eEF2K-KO, P=0.029, WT: n=10, eEF2K-KO: n=10; FIGS. 1F and 1G, FIG. 6G). There was no difference in cortical decorin levels between the groups (FIGS. 6H and 6K). These data suggest that the eEF2K/eEF2 pathway is upstream of neurogenesis in the mature DG.

The Inventors thus tested the hypothesis that the eEF2K/eEF2 pathway affects hippocampal neurogenesis and neurogenesis-dependent context discrimination. The inventors labeled dividing cells in two-month-old eEF2K-KO mice and WT littermates using BrdU. eEF2K-KO mice showed increased levels of proliferation (BrdU⁺ cells) in the granular and sub-granular zones in the dentate gyms compared to WT mice (T₁₄=−6.108, WT versus eEF2K-KO, P=0.000027, WT: n=8, eEF2K-KO: n=8; FIGS. 1H and 1I). Similarly, the inventors found a marked increase in adult-born neurons in eEF2K-KO compared to WT mice as measured by DCX immunohistochemistry (T₁₄=−9.283, WT versus eEF2K-KO, WT: n=8, eEF2K-KO: n=8, P=0.00000232; FIGS. 1J and 1K).

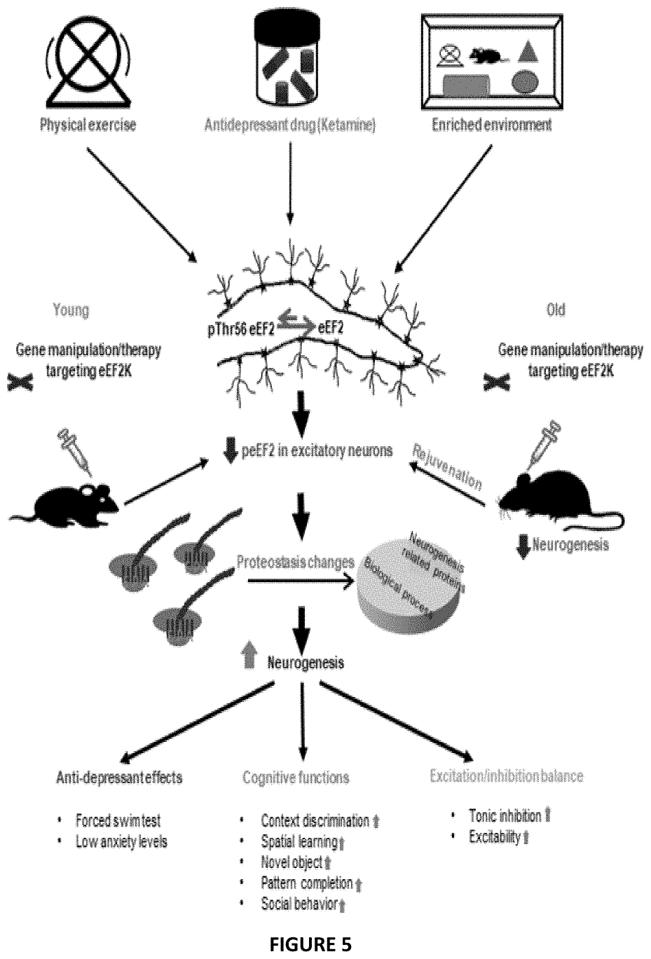

Levels of neurogenesis are known to correlate with specific forms of cognitive abilities. The inventors thus investigated whether increased hippocampal neurogenesis observed in eEF2K-KO mice correlated with DG-dependent behavior and subjected eEF2K-KO mice and WT littermates to the context discrimination of fear conditioning paradigm. eEF2K-KO and WT littermate mice showed comparable levels of freezing during acquisition of context A on days 1-3 (NP-ANOVA, Friedman test, χ²₂=36.333, WT versus eEF2K-KO, WT: n=11, eEF2K-KO: n=13, P<0.0001; FIGS. 1L-1M). On day 4, 2 weeks, and 4 weeks later, eEF2K-KO mice were able to distinguish between two highly similar contexts (A and B), whereas WT mice did not (Day 4 analysis: WT: ANOVA: model: F_{3.40}=7.563, P<0.0001, A versus B: P=0.726, A versus C: P=0.043, A versus D: P<0.0001; eEF2K-KO: NP-ANOVA, Kruskal-Wallis Test, χ²₃=25.771, P<0.0001, A versus B: U=18.500 P=0.001, A versus C: T₂₄=5.765 P<0.0001, A versus D: T₂₄=8.225 P<0.0001, Day 18 analysis: NP-ANOVA, Kruskal-Wallis test (all groups together), χ²₃=10.028, P=0.018, WT: A versus B: T₂₀=0.724 P=0.477, eEF2K-KO: A versus B: U=25.000, P=0.002, Day 32 analysis: ANOVA: model: F_{3.44}=3.725, P=0.018, WT: T₂₀=0.132, A versus B: P=0.897, eEF2K-KO: T₂₄=2.365, A versus B: P=0.026, WT A versus eEF2K-KO A: T₂₂=−2.330, P=0.029; eEF2K-KO: n=13, WT: n=11 in all days; FIGS. 1N-1P). Importantly, eEF2K-KO mice exhibited stronger contextual fear memory 4 weeks post-training. Both groups exhibited comparable levels of freezing in contexts C and D, which were markedly different from context A and B. Discrimination index between groups across days is shown in FIGS. 1M-10. eEF2K-KO mice showed significantly higher discrimination index on day 4 and day 32. Together, these results demonstrated that the eEF2K/eEF2 pathway controls both neurogenesis and DG-dependent pattern separation.

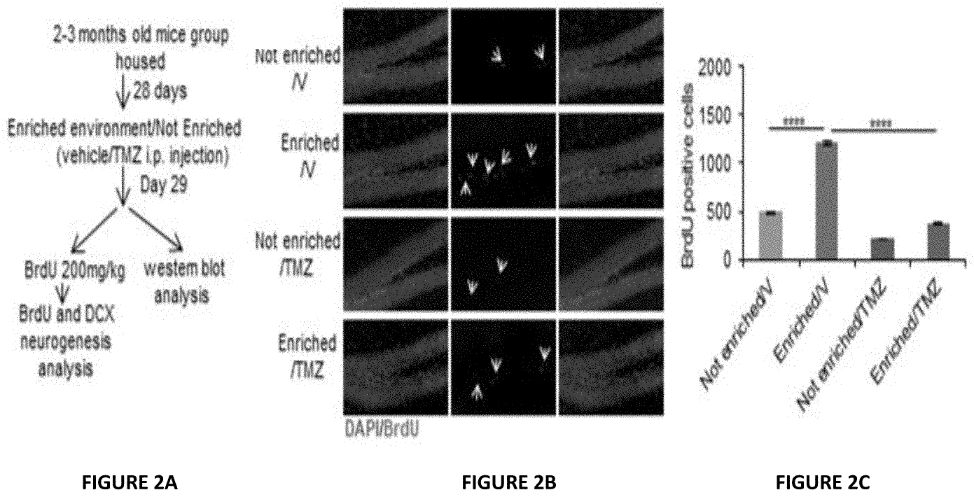

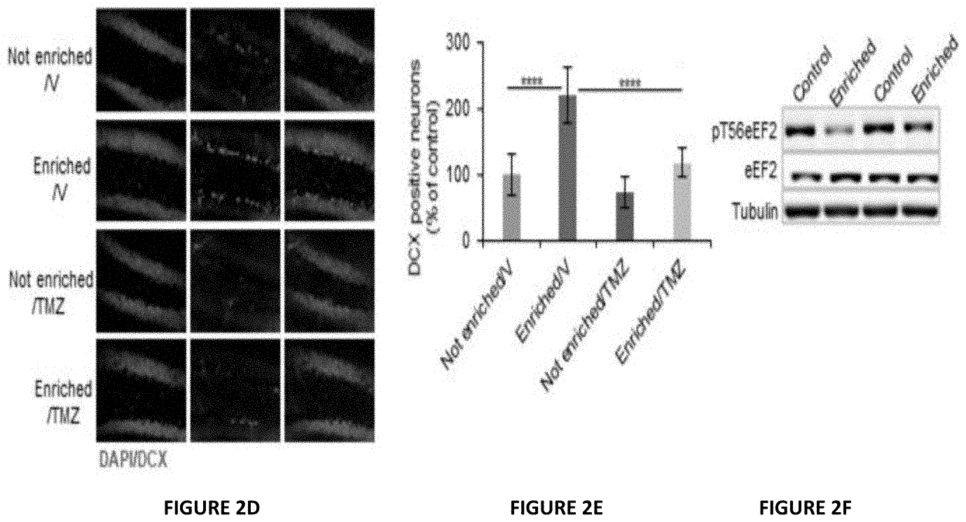

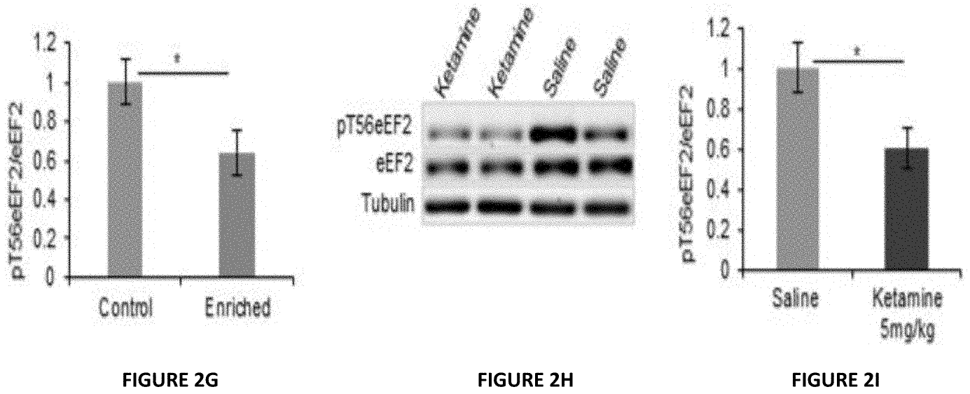

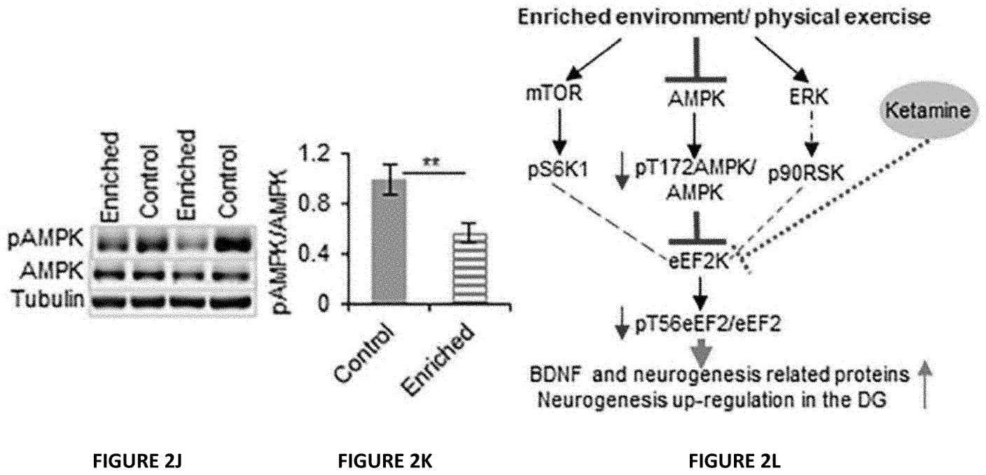

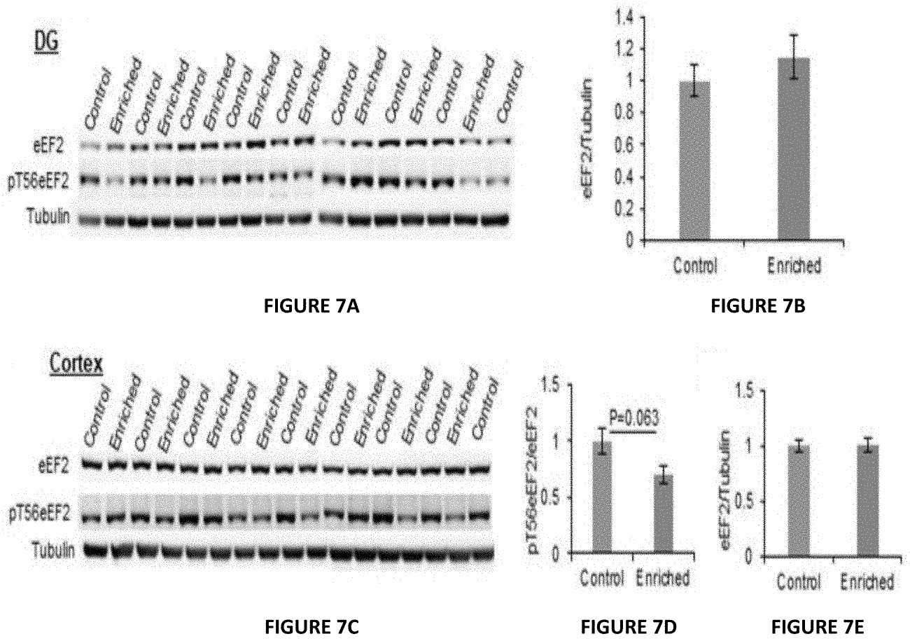

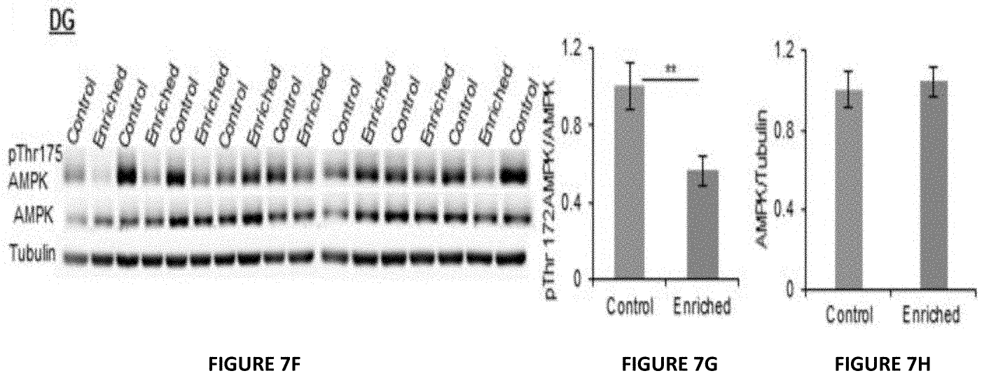

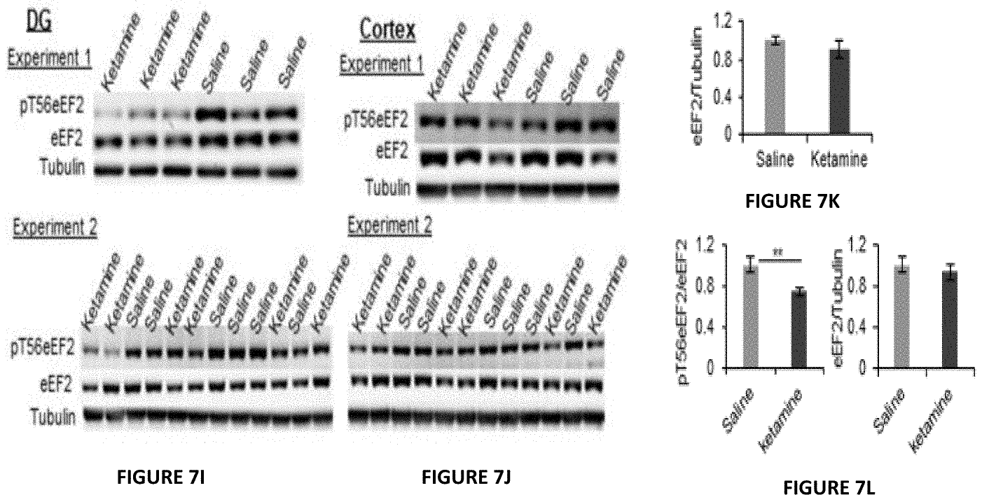

Next, the inventors explored whether the known physiological ways to promote adult hippocampal neurogenesis using enriched environment and voluntary exercise induced correlative eEF2 dephosphorylation in the DG. In agreement with the literature, exposure of naive mice to an enriched environment and voluntary exercise enhanced the number of BrdU-labeled cells in the DG compared to mice kept under standard conditions (FIG. 2A). This proliferation enhancement was precluded by administering temozolomide (TMZ; suppressor of adult neurogenesis, (Two-way ANOVA: model: F_{3.28}=39.856, P<0.0001; enriched/not enriched: F_{1.28}=40.090, P<0.0001, vehicle/TMZ: F_{1.28}=62.661, P<0.0001, interaction: F_{1.28}=16.816, P<0.0001; not enriched/V versus enriched/V: T₁₄=−5.759, P<0.0001, enriched/V versus enriched/TMZ: T₁₄=6.842, P<0.0001; n=8 mice in each group; FIGS. 2B-2C). Similarly, exposure to an enriched environment and voluntary exercise enhanced adult-newborn neurons in the DG as labeled by DCX, and this increase was blocked by TMZ treatment (Two-way ANOVA: model: F_{3.28}=28.922, P<0.0001; enriched/not enriched: F_{1.28}=47.873, P<0.0001, vehicle/TMZ: F_{1.28}=29.204, P<0.0001, interaction: F_{1.28}=9.690, P=0.004; Not enriched/V versus enriched/V: T₁₄=−5.893, P<0.0001, enriched/V versus enriched/TMZ: T₁₄=5.528, P<0.0001; n=8 mice in each group; FIGS. 2D-2E). In line with the enhanced neurogenesis, levels of peEF2 were significantly decreased in the DG following exposure to an enriched environment and voluntary exercise (Mann-Whitney Test: U=14.000, P=0.034; enriched group=8 mice, not enriched group=9 mice; FIGS. 2F-2G, and FIG. 7A). There was no effect on eEF2 levels in the DG or peEF2 levels in the cortex (FIG. 7B-7E). In addition, peEF2 levels were reduced 30 min following ketamine injection, which is known to induce neurogenesis and work as an anti-depressant in the DG (Mann-Whitney Test, U=0.000, P=0.050; nₛₐₗᵢₙₑ=3, nₖₑₜₐₘᵢₙₑ=3, FIGS. 2H-2I, and FIGS. 7I-7K). Together, these findings suggest that enriched environment, physical exercise, and ketamine converge on eEF2K in the DG to increase neurogenesis.

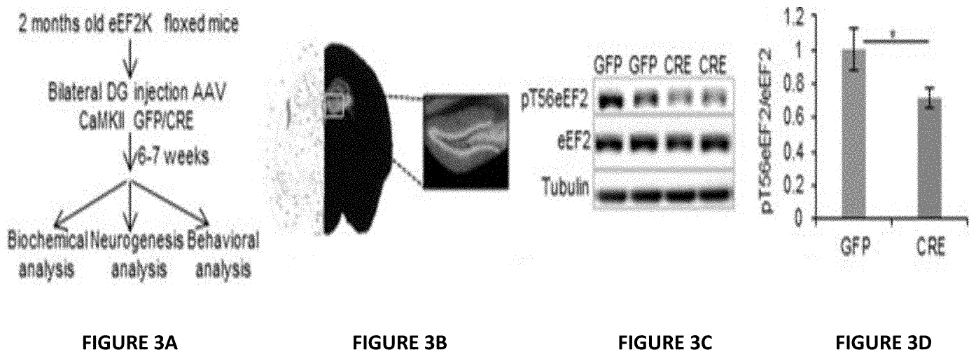

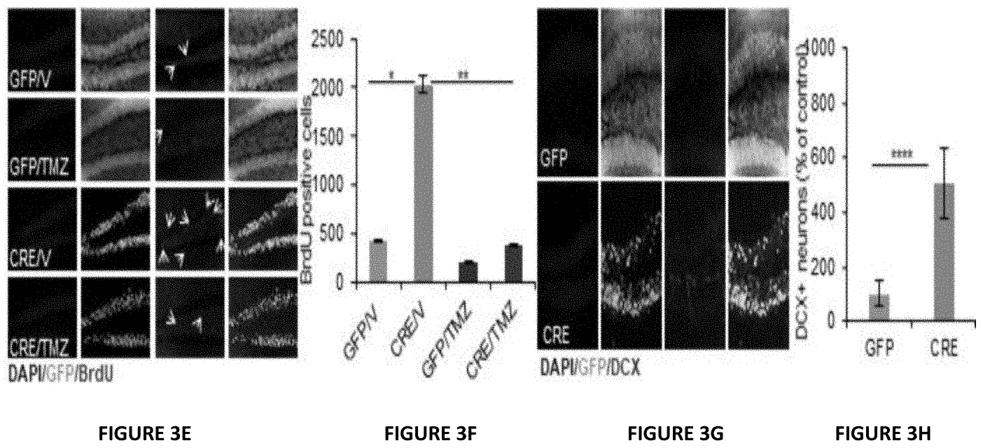

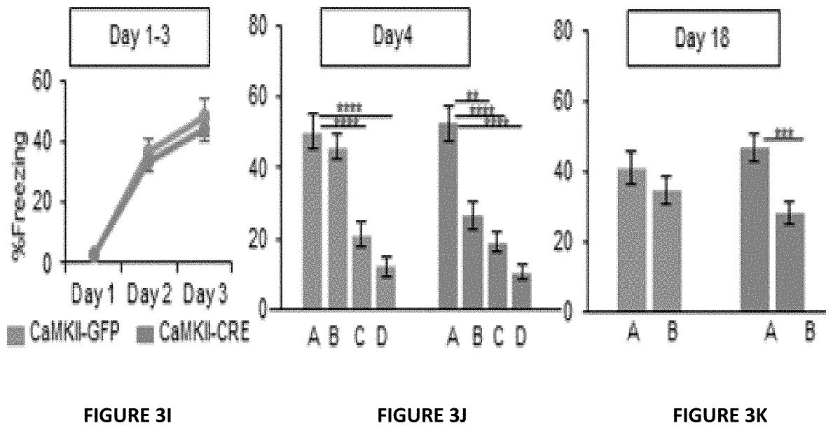

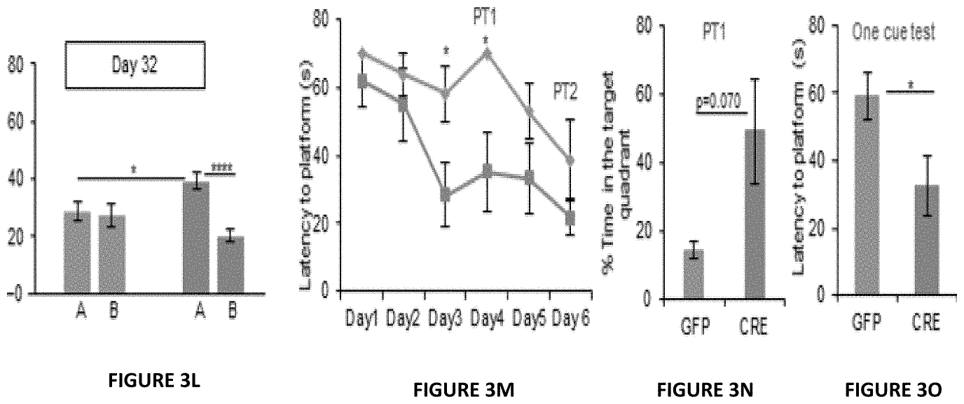

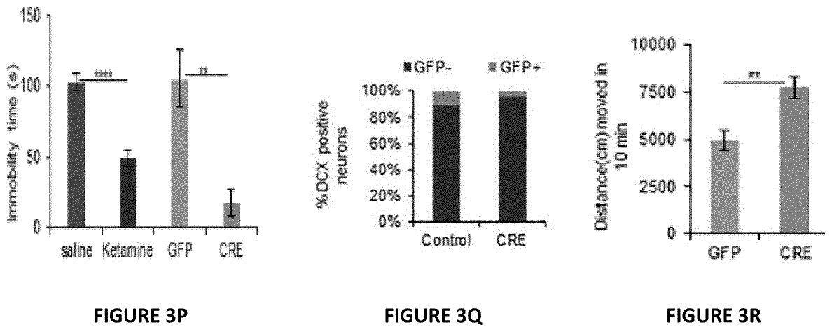

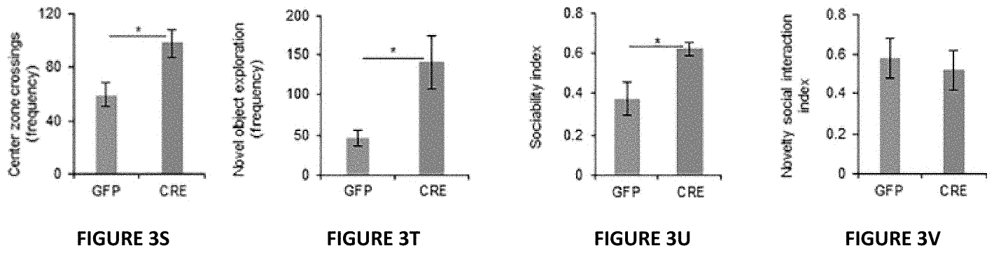

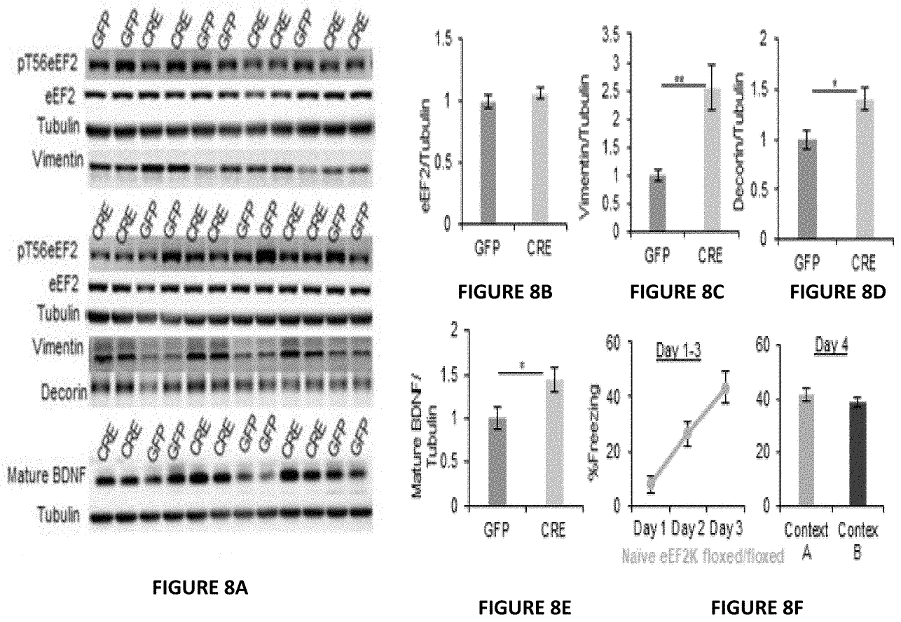

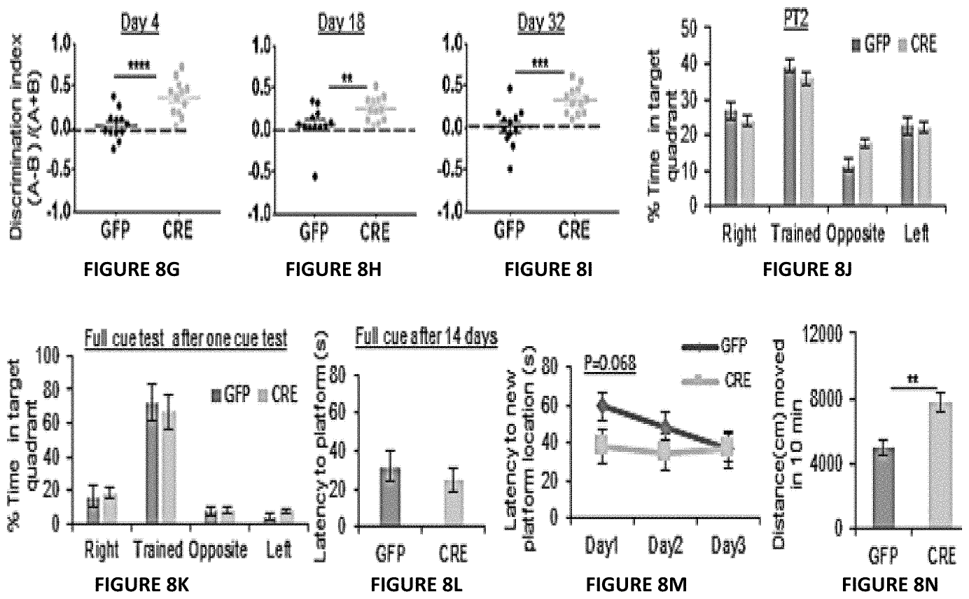

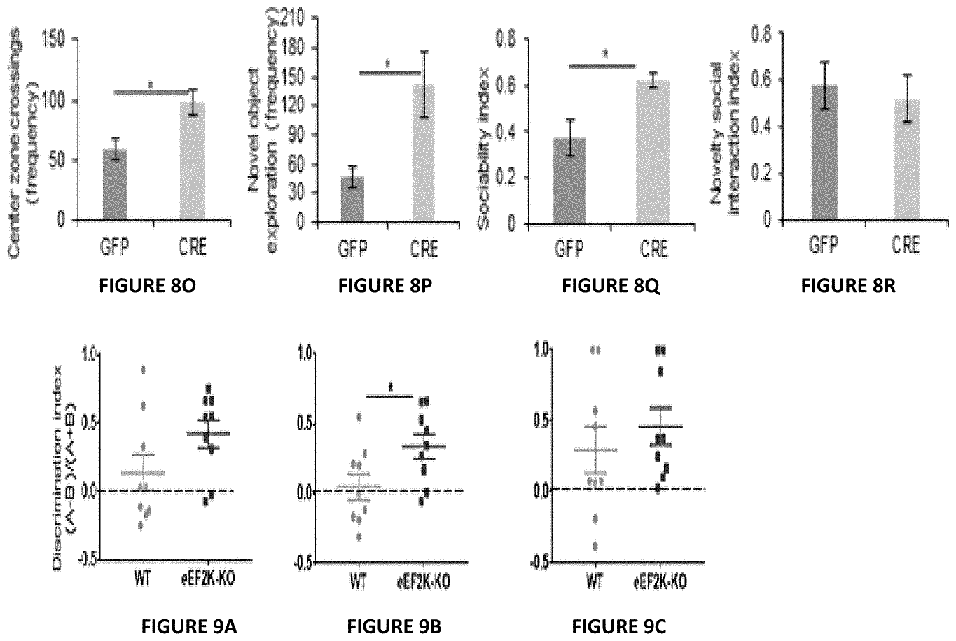

The inventors thus tested the hypothesis that genetic reduction of eEF2 phosphorylation specifically in mature excitatory neurons in the DG of mature mice will enhance neurogenesis and neurogenesis-dependent phenotypes. To do so, the inventors generated transgenic eEF2K homozygote floxed/floxed mice, where eEF2K was flanked by lox p sites and was excised upon AAV-CaMKII-Cre and GFP expression in mature excitatory neurons. AAV (serotype: AAV1, Penn Vector Core, University of Pennsylvania)-CaMKII-Cre or GFP were bilaterally injected to the DG of 2-month-old mice, and 6-7 weeks later, the inventors examined the effect of reducing eEF2K levels on behavior, neurogenesis, and biochemistry in the DG (FIGS. 3A-3B). The inventors observed a significant reduction in peEF2 levels normalized to eEF2 in the DG of CaMKII-Cre injected mice compared to CaMKII-GFP-injected mice (Mann-Whitney U test, U=1, P=0.016, ncᵣₑ=5, n_{GFP}=5; FIGS. 3C-3D, and FIG. 8A) with no change in eEF2 protein levels (FIGS. 8A-8B). First, the inventors compared the molecular changes in CaMKII-Cre injected mice. Similarly, to null eEF2K-KO mice (FIG. 1), decorin and vimentin levels were up-regulated in the dentate gyms of CaMKII-Cre injected mice compared to GFP-injected mice (FIGS. 8A, and 8C). Importantly, BrdU-labeled cells were increased in CaMKII-Cre-injected mice compared to CaMKII-GFP-injected mice, which was precluded by TMZ (Two-way ANOVA: model: F_{3.32}=9.357, p<0.0001, Genetic manipulation(CRE/GFP): F_{1.32}=10.267, P=0.003, Treatment(vehicle/TMZ): F_{1.32}=11.223, P=0.002, interaction: F_{1.32}=6.580, P=0.015; GFP/V versus CRE/V: T_{8.436}=−2.958, P=0.017, CRE/V versus CRE/TMZ: Mann-Whitney U test, U=6.000, P=0.002; n=9 in each group, FIGS. 3E and 3F). In addition, CaMKII-Cre-injected mice showed significantly higher levels of DCX⁺ neurons compared to CaMKII-GFP-injected mice (T₁₆=−7.043, P<0.0001, n=9 mice in each group; FIGS. 3G and 3H). Together, these observations demonstrate that mice with reduced eEF2K levels in mature excitatory neurons in the DG have a similar molecular phenotype to general eEF2K-KO mice, and this molecular change is sufficient to promote neurogenesis in the DG.

To determine whether the enhanced adult hippocampal neurogenesis observed in CaMKII-Cre-injected mice correlates with cognitive improvements, the inventors performed a series of hippocampal-dependent behavioral tests. First, naïve floxed/floxed mice were conditioned in context A for three days. Naïve eEF2K floxed/floxed mice and CaMKII-GFP-injected mice showed comparable levels of context A acquisition for three days (FIG. 8D). In addition, there was no difference between naive eEF2K floxed/floxed and CaMKII-GFP-injected mice in discrimination between context A and B on day 4 (FIG. 8E). Next, young (3-month-old) CaMKII-Cre-injected mice and CaMKII-GFP-injected mice underwent context discrimination of fear conditioning for one month as shown in FIG. 1L. CaMKII-Cre-injected mice and CaMKII-GFP-injected mice showed comparable levels of acquisition of contextual fear learning on days 1-3 (NP-ANOVA, Friedman test, χ²₂=40.083, P<0.0001, GFP: n=12, CRE: n=12; FIG. 3I). Interestingly, on day 4, 2 weeks, and 4 weeks, CaMKII-Cre-injected mice were able to discriminate between two highly similar contexts A and B, whereas the CaMKII-GFP-injected mice could not distinguish between them (Day 4 analysis: GFP: NP-ANOVA, Kruskal-Wallis Test, X²₃=31.899, P<0.0001, A versus B: T₂₂=0.663, P=0.514, A versus C: U=9, P<0.0001, A versus D: U=3, p<0.0001; CRE: NP-ANOVA, Kruskal-Wallis Test, χ²₃=28.572, P<0.0001, A versus B: U=17 P=0.001, A versus C: U=4, P<0.0001, A versus D: U=0, P<0.0001, Day 18 analysis: One-way ANOVA, (all groups together), F_{3.44}=4.029, P=0.013, GFP: A versus B: T₂₂=1.049 P=0.305, CRE: A versus B: T₂₂=3.689, P=0.001, Day 32 analysis: One-way ANOVA: model: F_{3.44}=6.137, P=0.001, GFP: A versus B, T₂₂=0.295, P=0.771, CRE: A versus B: T₂₂=5.233, P<0.001, GFP A versus CRE A: T₂₂=−2.337, P=0.029; CRE: n=12, GFP: n=12 in all days; FIGS. 3J-3L). Importantly, CaMKII-Cre-injected mice showed stronger contextual fear memory 4 weeks post-training. CaMKII-Cre-injected mice and CaMKII-GFP-injected mice exhibited comparable levels of freezing in contexts C and D. In addition, CaMKII-Cre-injected mice showed significantly higher discrimination index after one month (FIGS. 8F-8H).

To support and extend the findings obtained in the context discrimination paradigm, the inventors subjected mice to a weak protocol of the Morris water maze. CaMKII-Cre-injected mice located the hidden platform significantly faster than CaMKII-GFP-injected mice during the acquisition phase (days 1-6) of the Morris water maze (NP-ANOVA, Friedman test, χ²₅=24.857, P<0.0001, GFP: n=6, CRE: n=6, Day 3: GFP versus Cre: Mann-Whitney U test, U=6, P=0.046, Day4: GFP versus Cre: Mann-Whitney U test, U=6, P=0.022; FIG. 3M). Since the inventors observed significant differences in escape latencies on day 3, the first probe test (PT1) was performed on day 4. CaMKII-Cre-injected mice spent slightly more time in the target quadrant compared to CaMKII-GFP mice (GFP versus Cre: T_{5.290}=−2.261, P=0.070; FIG. 3N). In addition, the second probe test (PT2) was performed on day 6 after completing the training. CaMKII-Cre- and CaMKII-GFP-injected mice similarly preferred the target quadrant (FIG. 8I). Later, the inventor assessed reversal of spatial learning by switching the platform to the opposite quadrant. In the new location, CaMKII-Cre-injected mice located the platform slightly faster than CaMKII-GFP-injected mice (FIG. 8L), suggesting slightly enhanced cognitive flexibility. Adult-born DG neurons mediate pattern separation, whereas old granule neurons facilitate pattern completion. Therefore, the inventors tested the effect of manipulating the eEF2K/eEF2 pathway on pattern completion as well. Two additional cohorts of mice were trained in the Morris water maze; two weeks after the last probe test, both groups were subjected to one cue probe test in which three navigation cues were removed, and the pool was left with one cue. The latency of CaMKII-Cre-injected mice was significantly lower compared to CaMKII-GFP-injected mice (GFP versus Cre: U=6, P=0.046, FIG. 30). Following partial cues, another full cue test was performed. CaMKII-Cre- and CaMKII-GFP-injected mice had comparable latencies to locating the platform and spent similar time in the training quadrant (FIGS. 8J-8K). Together, these observations demonstrate that reduced eEF2K levels in mature excitatory neurons of the DG facilitate both pattern separation of contextual fear memory and spatial pattern completion.

In addition to the enhanced DG dependent cognitive abilities, the inventors looked at 3 more behavior phenotypes known to be controlled by neurogenesis in the DG: anxiety, social behavior, and depression like behavior. Adult-hippocampal neurogenesis was inversely correlated with anxiety-like behavior. Thus, the inventors measured anxiety-like behavior in the open field arena paradigm. CaMKII-Cre-injected mice crossed the center zone and travelled more in the open field arena compared to CaMKII-GFP-injected mice, suggesting decreased anxiety-like behavior (FIGS. 8M-8N). In addition, CaMKII-Cre-injected mice better identified objects as familiar in the object recognition task, manifested in shorter times spent exploring the familiar object compared to CaMKII-GFP-injected mice (FIG. 8O). Since hippocampal neurogenesis levels are correlated with oxytocin levels in the DG, and oxytocin receptor signaling in the DG is necessary for social discrimination, the inventors performed classical social behavior tests, in which the mice underwent social interaction and novelty social tests. CaMKII-Cre-injected mice showed significantly higher sociability index compared to CaMKII-GFP-injected mice (FIG. 8P). In addition, CaMKII-Cre-injected mice and CaMKII-GFP-injected mice showed comparable novelty social interaction indices (FIG. 8Q). Another DG neurogenesis-dependent behavior is depression. The inventors therefore subjected CaMKII-Cre-injected and CaMKII-GFP-injected mice to the forced swim test. As a positive control, the inventors injected ketamine (5 mg/kg i.p.) to WT naïve mice. CaMKII-Cre-injected mice showed significantly lower immobility time compared to CaMKII-GFP-injected mice. As expected, ketamine-treated WT mice exhibited significantly lower immobility time compared to saline-treated WT mice (NP-ANOVA, Kruskal-Wallis Test, χ²₃=15.644, P=0.001, Cre versus GFP: U=2, P=0.009, saline versus ketamine, T₁₁=6.490 P<0.0001; FIG. 3P). Taken together, our data provide evidence for the critical role of the eEF2K/eEF2 pathway-dependent mRNA translation elongation in the mature DG in pattern separation, spatial memory, pattern completion, social interaction, and depression like behavior.

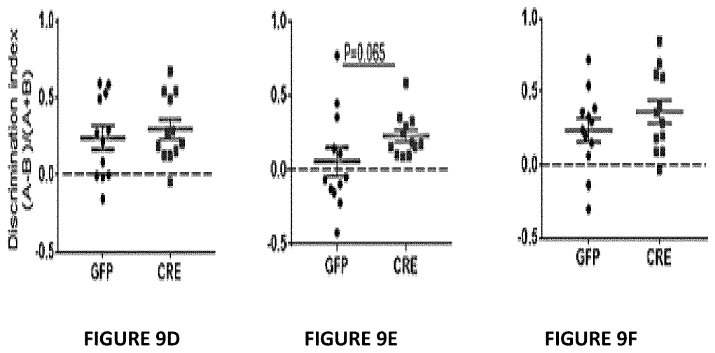

Brain slices from eEF2K ko mice exhibit enhanced tonic inhibition in the DG, the inventors thus examined the effect of conditional eEFK2 deletion on basal synaptic transmission and long-term potentiation (LTP) in the perforant path input to dentate gyms of anesthetized mice. There were no differences on the synaptic transmission or the induction of early phase LTP (FIG. 9). However, in striking contrast to CaMKII-GFP control mice which exhibit normal stable LTP following massed HFS, in CaMKII-Cre injected mice, an initial increase in the fEPSP was following by decline to baseline levels at 50 min followed by a slow rise to reach the same LTP magnitude as control at 3 hours post-HFS. Thus, removal of eE2K enhances tonic inhibition and modulates the dynamics of LTP expression but did not alter LTP induction or the generation of persistent late LTP. Given the pronounced enhancement of neurogenesis after removing eEF2K, these suggested that synapses of new neurons have distinct LTP expression dynamics or alter the property of pre-existing perforant path synapses.

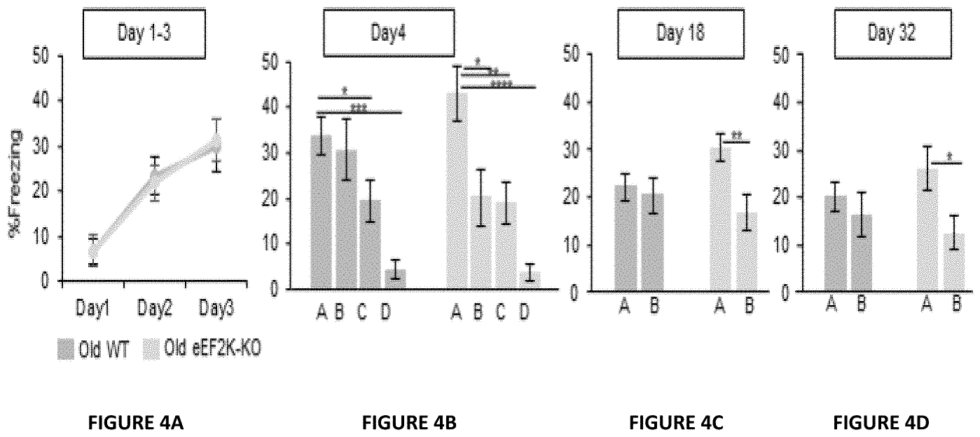

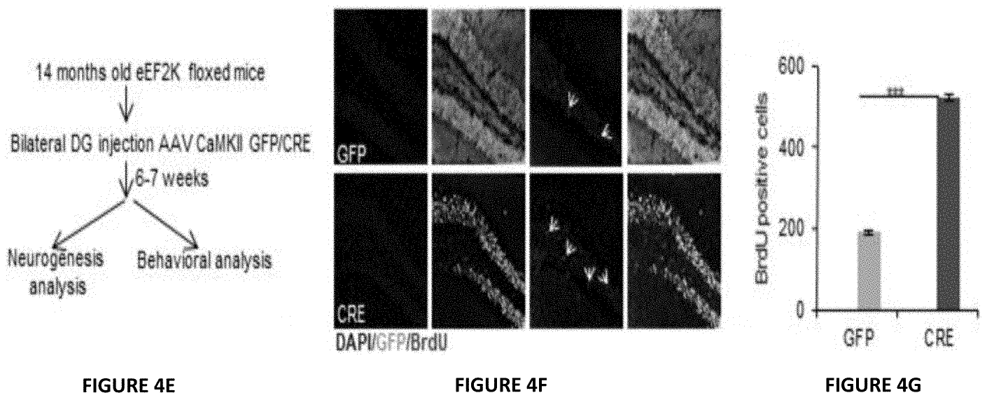

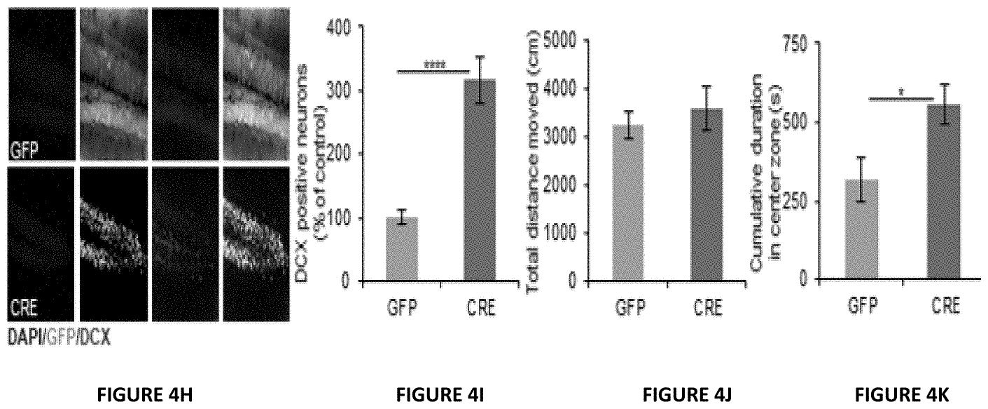

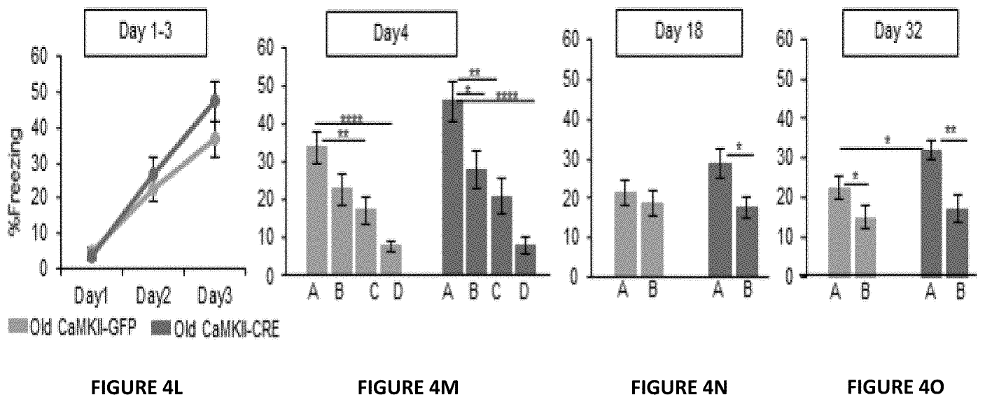

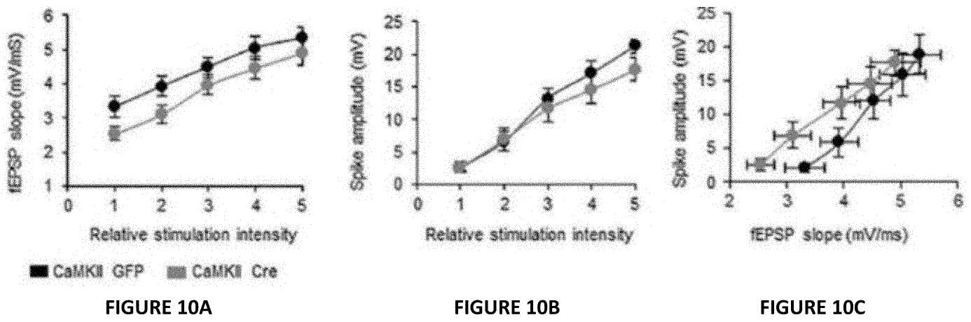

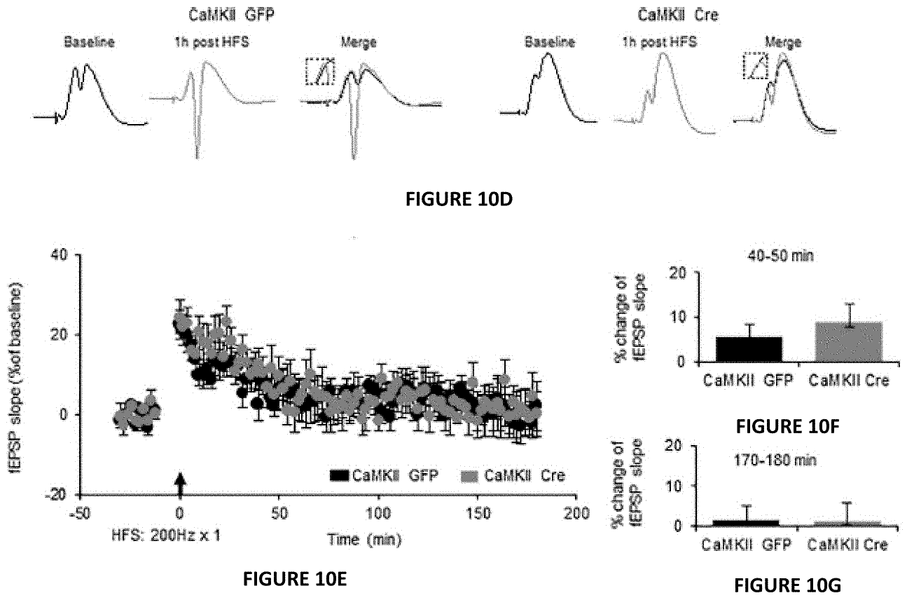

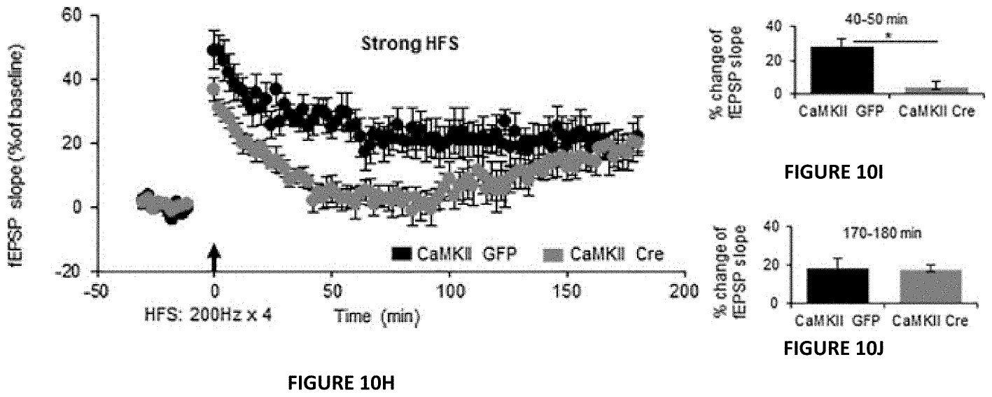

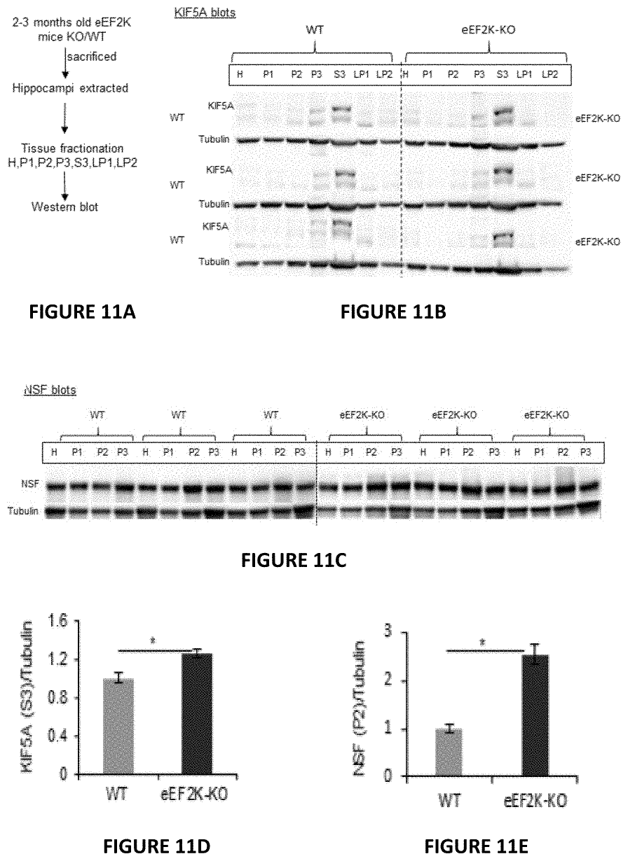

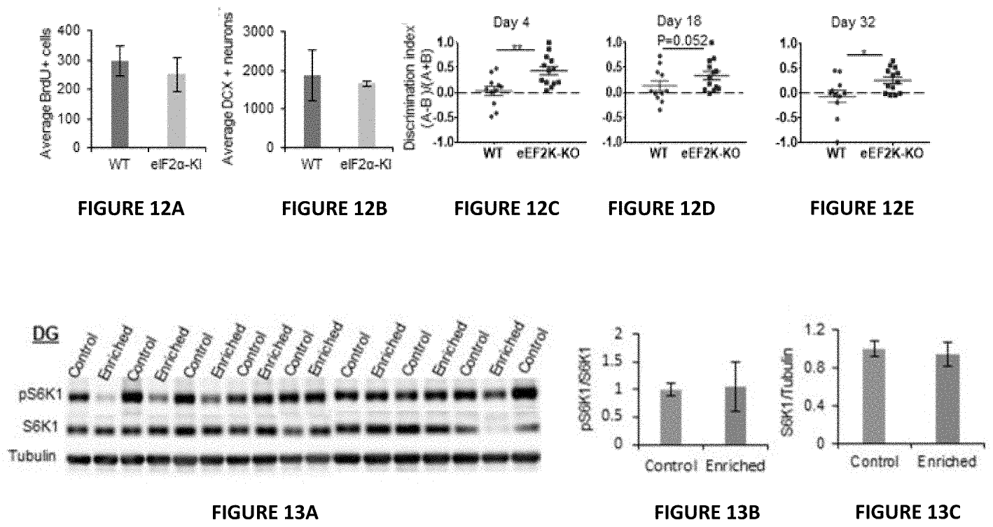

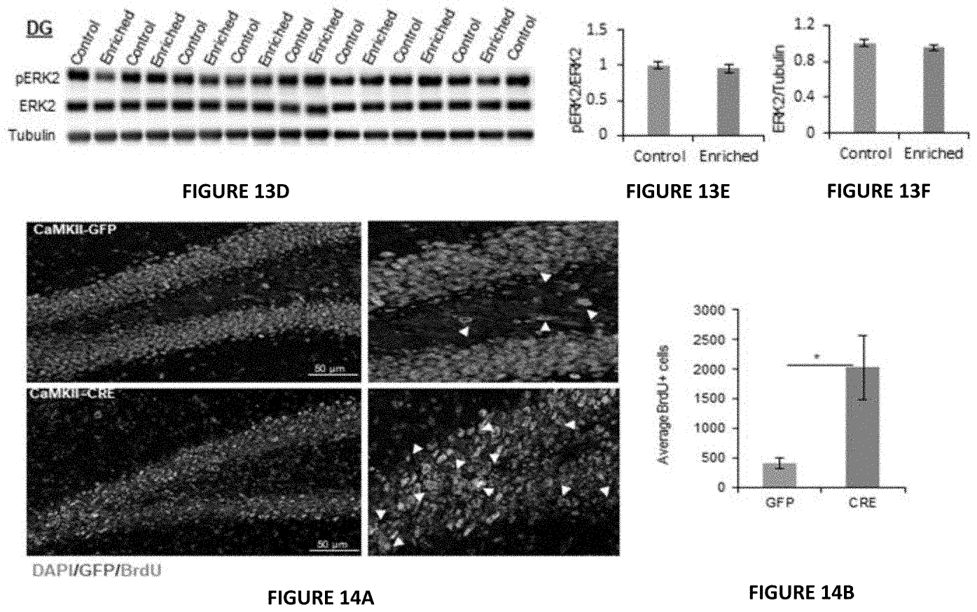

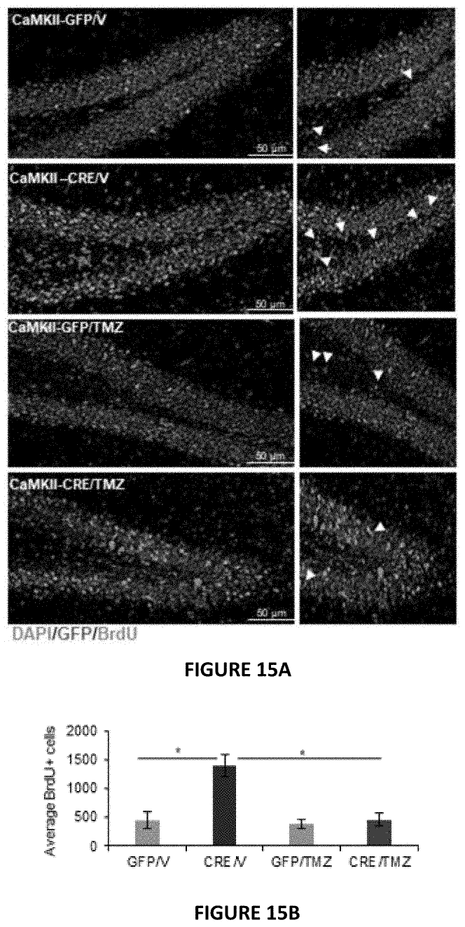

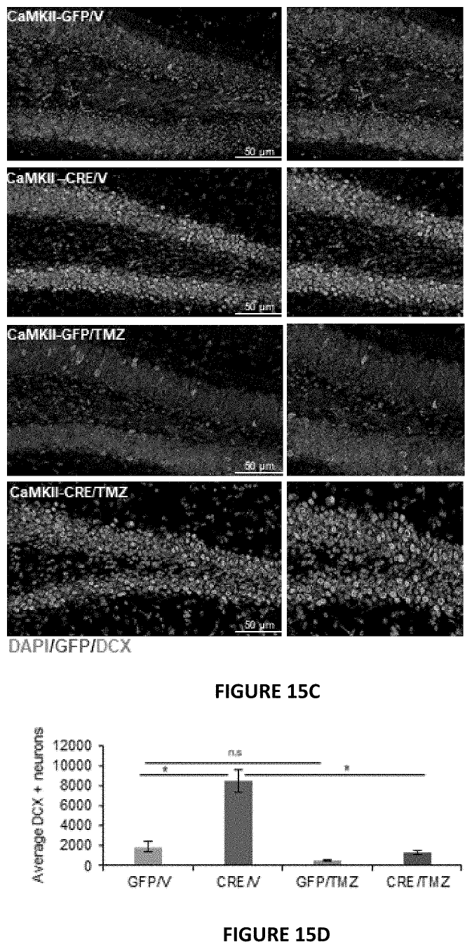

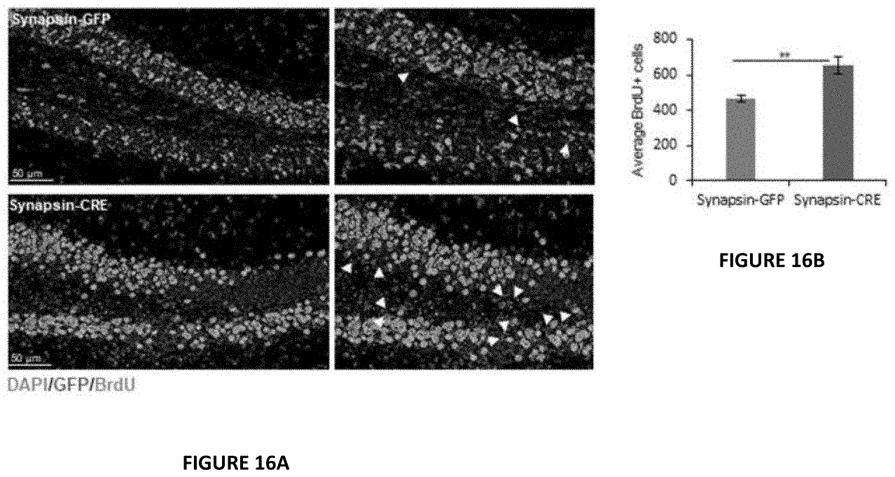

Aging was linked to reduced cognitive abilities and sharply decreased neurogenesis in the hippocampal DG and other neurogenic regions in the brain of humans and other mammalians. Thus, the inventors next examined the possibility of rejuvenating the old DG by reducing eEF2 phosphorylation levels in the old DG. Towards that aim, 15-16-month-old eEF2K-KO mice and WT littermates underwent context discrimination for one month. Old eEF2K-KO and WT mice showed comparable levels of freezing in context A acquisition on days1-3 (NP-ANOVA, Friedman test, χ²₂=27.444, P<0.0001, FIG. 9A). On day 4, 2 weeks, and 4 weeks later, eEF2K-KO old mice were able to distinguish between two highly similar contexts (A and B), whereas the WT found it difficult to discriminate between the two contexts (Day 4 analysis: WT: NP-ANOVA, Kruskal-Wallis Test, χ²₃=17.068, P=0.001, A versus B: T₁₆=0.423, P=0.678, A versus C: T₁₆=2.310, P=0.035, A versus D: U=2, P=0.001; eEF2K-KO: NP-ANOVA, Kruskal-Wallis Test, χ²₃=20.767, P<0.0001, A versus B: U=13, P=0.015, A versus C: T₁₆=3.165, P=0.006, A versus D: U=0, P<0.0001, Day18 analysis: NP-ANOVA, Kruskal-Wallis Test (all groups together), χ²₃=9.358, P=0.025, WT: A versus B: U=38, P=0.825, eEF2K-KO: T₁₆=2.915, P=0.010, Day 32 analysis: One-way ANOVA: model: F_{3.32}=1.999, P=0.134, WT: A versus B, T₁₆=0.708, P=0.489, eEF2K-KO: A versus B: T₁₆=2.249, P=0.039, WT A versus eEF2K-KO A: T₁₆=−1.026, P=0.320; WT: n=9, eEF2K-KO: n=9 in all days, FIGS. 4B-4D). Both groups exhibited comparable levels of freezing in contexts C and D. Discrimination indices between groups across days is shown in FIGS. 10A-10C. Old eEF2K-KO mice showed significantly higher discrimination index on day18. Since it is possible that the enhanced cognitive abilities in the old eEF2K-KO was pre-determined in young age, the inventors examined the possibility of reversing the reduced hippocampal neurogenesis and related cognitive functions in old mice by specific reduction of eEF2K in the DG in 14-month-old mice. Naïve (14-month-old) floxed/floxed old mice were bilaterally injected via the DG with AAV encoding CaMKII-Cre or CaMKII-GFP. 6-7 weeks later they were subjected to the open field test and context discrimination paradigm, and later then sacrificed for neurogenesis analysis (FIG. 4E). Strikingly, CaMKII-Cre-injected old mice exhibited significantly increased levels of BrdU-labeled cells and DCX-labeled neurons in the DG compared to old CaMKII-GFP-injected mice (BrdU analysis: GFP versus Cre: Mann-Whitney U test, U=3, P<0.001, DCX analysis: GFP versus Cre: T_{10.011}=−7.465, P<0.0001; n=9 in each group, FIG. 4F-4I). CaMKII-Cre-injected old mice and CaMKII-GFP-injected old mice showed comparable levels of distance travelled in the open field test. However, CaMKII-Cre-injected old mice spent significantly more time in the center zone of the open field (total distance moved: GFP versus Cre, T₁₀=−0.639, P=0.537; Time in center zone: GFP versus Cre, T₁₀=−2.580, P=0.027, n=6 in each group, FIGS. 4J-4K), suggesting decreased anxiety-like behavior. Next, the inventors aimed to investigate whether increased hippocampal neurogenesis in old mice results also in memory enhancement. CaMKII-Cre-injected mice and CaMKII-GFP-injected mice showed comparable acquisition of contextual fear learning on days 1-3 (NP-ANOVA, Friedman test: χ²₂=42.750, p<0.0001; FIG. 4L). Of note, on day 4, 2 weeks, and 4 weeks later, CaMKII-Cre-injected old mice were able to discriminate between the two highly similar contexts A and B, whereas CaMKII-GFP-injected old mice could not distinguish between them (Day 4 analysis, GFP: Kruskal-Wallis Test, χ²₃=20.065, p<0.0001, A versus B: T₂₂=1.867, P=0.075, A versus C: T₂₂=3.025, P=0.006, A versus D: T_{13.283}=5.971, P<0.0001; Cre: Kruskal-Wallis Test: χ²₃=22.497, P<0.0001, A versus B: T₂₂=2.577, P=0.017, A versus C: T₂₂=3.612, P=0.002, A versus D: U=2, P<0.0001, Day18 analysis: Kruskal-Wallis Test, χ²₃=6.704, P=0.082, GFP: A versus B: T₂₂=0.605, P=0.551, Cre: A versus B: U=34, P=0.028, Day 32 analysis: Kruskal-Wallis Test, χ²₃=13.969, P=0.003, GFP: A versus B: U=33, P=0.024, Cre: A versus B: T₂₂=3.371, P=0.003, GFP A versus Cre A: T₂₂=−2.534, P=0.019; GFP: n=12, Cre: n=12 in all days, FIGS. 4M-4O). Importantly, CaMKII-Cre-injected old mice showed stronger contextual fear memory four weeks post-training. CaMKII-Cre-injected mice and CaMKII-GFP-injected old mice exhibited comparable levels of freezing in contexts C and D. Discrimination index between groups across days was shown in FIGS. 10D-10F. CaMKII-Cre-injected old mice showed non-significant increase in discrimination index on day 18. These observations clearly demonstrate that manipulating the eEF2K/eEF2 pathway in the DG of old mice rejuvenates hippocampal neurogenesis and cognitive abilities and improves long-term memory.

Adult neurogenesis in the mammalian brain was proposed 60 years ago but met substantial resistance and skepticism from the scientific community since it contradicted the long-held dogma. Neurogenesis is currently a subject of intense research in humans and other species with clear correlations between levels of neurogenesis in the mature DG and better learning in different cognitive tasks, though some studies suggests that the ability to retrieve old memories may decline at the same time. The process of the transition from stem cell to neuronal progenitors and later to newborn young neurons can be divided into five stages that are mediated by molecular signals operating in the highly specific DG niche (l) Levels of neurogenesis in the mature DG are behaviorally linked to archetypic behavior (e.g.) and mental states (e.g.,) and electro-physiologically to excitation/inhibition (E/I) balance.

A pivotal question in the field is whether it is possible to reverse the age-related decline in neurogenesis. Answering this question requires better identification of the molecular pathways that control the different phases of mature neurogenesis. It is known that enriched environment, physical activity, or ketamine administration can enhance neurogenesis but the biological machinery underlying these processes is unclear. Here, the inventors identified the eEF2/eEF2K pathway as a master regulator of neurogenesis in the mature DG and related phenotypes. Moreover, the inventors show that the other physiological ways to increase neurogenesis converge on the eEF2K pathway.

In contrast to eEF2-KI mice, with residual phosphorylation of eEF2 and complex phenotype, in the eEF2K-KO mice used in the current study, eEF2 was constantly dephosphorylated and thus the increased rates of translation elongation of mRNA transcripts enable changes the proteome. Surprisingly, this enhanced rate of mRNA elongation enhanced the expression of many proteins underlying neurogenesis, differentiation, and growth (FIG. 1 and FIG. 6). However, the fact that identical phenotypes were observed using the full eEF2K-KO and the CaMKII-driven KO, in which eEF2 phosphorylation is decreased in mature excitatory granules cells in the DG, suggests that signals to promote proliferation, as indicated by BrdU, and differentiation and maturation, as indicated by DCX, are occurring mainly in mature excitatory neurons. Interestingly, dopamine, one of the few known molecular cues in the mature DG to signal for proliferation is upstream of eEF2 dephosphorylation in neurons. Furthermore, both Wnt/PCP and BDNF, two of the very few signals to induce morphogenesis and differentiation in the mature DG, are affected by eEF2 pathway regulation.

It is clear that neurogenesis in the DG of the adult hippocampus declines sharply with age and is affected by the lifespan of the studied species. However, calculating the numbers of newly in cooperated neurons over 10 years, results in highly meaningful numbers. In addition, reduced neurogenesis is linked to different neurodegenerative and mental diseases. When the inventors enhanced eEF2 activity, in mature excitatory DG neurons of old mice, they restored neurogenesis and cognitive abilities to ‘younger-like’ levels. This opens the door in the future to rejuvenating the human hippocampus pharmacologically or genetically, and thus perhaps tackle both abnormal and normal age-related cognitive decline by modulating the eEF2K/eEF2 pathway.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating a neurological disease in a subject in need thereof, the method comprising administering to the subject a pharmaceutical composition comprising therapeutically effective amount of a eukaryotic elongation factor 2 kinase (eEF2K)-inhibiting compound, thereby treating a neurological disease in the subject.

2. The method of claim 1, wherein said treating comprises inducing neuron proliferation in said subject.

3. The method of claim 1, wherein said eEF2K-inhibiting compound inhibits eEF2K kinase activity.

4. The method of claim 1, wherein said eEF2K-inhibiting compound reduces the rate of eEF2 phosphorylation, the number of phosphorylated eEF2 molecules, or both.

5. The method of claim 1, wherein said eEF2K-inhibiting compound is selected from the group consisting of: nucleic acids, peptides, polypeptides, peptidomimetics, carbohydrates, lipids or other organic or inorganic molecules.

6. The method of claim 2, wherein said neuron is a neuron of the hippocampus.

7. The method of claim 2, wherein said neuron is a neuron of the dentate gyms (DG).

8. The method of claim 6, wherein said neuron is a mature excitatory neuron.

9. The method of claim 2, wherein said neuron is a neuron of a subject afflicted with a neurological disease.

10. The method of claim 1, wherein said neurological disease is selected from the group consisting of: Alzheimer's disease, Parkinson's disease, dementia, depression, epilepsy, memory loss, and cognitive impairment.

11. (canceled)

12. (canceled)

13. A method for screening for a compound suitable for treating a neurological disease, the method comprising contacting a neuron with a compound, and determining the activity of eEF2K in the presence of said compound, wherein a reduction of eEF2K activity in said neuron in the presence of said compound compared to eEF2K activity in a neuron in the absence of said compound is indicative of said compound is suitable for treating a neurological disease.

14. The method of claim 13, wherein said eEF2K activity is ATP hydrolysis activity.

15. The method of claim 13, wherein said eEF2K activity is eEF2 phosphorylation.

16. The method of claim 15, wherein said phosphorylation is on Threonine 56 of said eEF2.

17. The method of claim 13, wherein said eEF2K activity is protein translation inhibition.
